(19) United States
(12) Patent Application Publication
(10) Pub. No.: US 2026/0356869 A1
(43) Pub. Date: Nov. 12, 2026

(54) TRANSITION METAL COMPLEXES FOR MRI APPLICATIONS AND METHODS OF USE

(71) Applicant: Yale University, New Haven, CT (US)

(72) Inventors: Sandeep Kumar Mishra, New Haven, CT (US); Muhammad Khan, New Haven, CT (US); ABM Zakaria, New Haven, CT (US); Jelena Mihailovic, New Haven, CT (US); Daniel Coman, New Haven, CT (US); Fahmeed Hyder, New Haven, CT (US)

(21) Appl. No.: 19/099,684

(22) PCT Filed: Oct. 31, 2023
(86) PCT No.: PCT/US2023/036363

§ 371 Date: Jan. 29, 2025

Related U.S. Application Data
(60) Provisional application No. 63/421,051, filed on Oct. 31, 2022.

(51) Int. Cl. C07F 15/02; A61K 49/10; C07F 15/04; C07F 15/06

(52) U.S. Cl. CPC C07F 15/025; A61K 49/106; C07F 15/045; C07F 15/065

(57) ABSTRACT

Provided herein are certain transition metal complexes of formula (Ia), (Ib), (Ic), (Id), or (Ie). The transition metal complexes are suitable for use as MRI contrast agents and can be used for diagnosing the prognosis of cancer by measuring the environment surrounding a cancerous tumor.

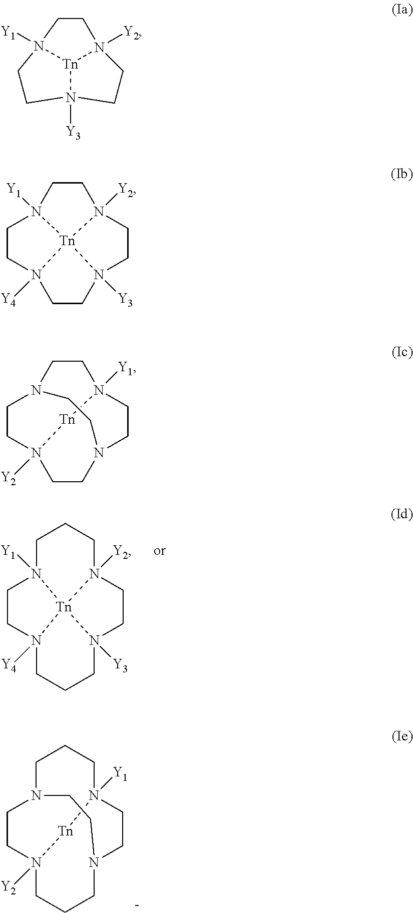

### CROSS-REFERENCE TO RELATED APPLICATION

This application claims priority to U.S. Provisional Patent Application No. 63/421,051 entitled “TRANSITION METAL COMPLEXES FOR MRI APPLICATIONS AND METHODS OF USE,” filed Oct. 31, 2022, the disclosure of which is incorporated herein by reference in its entirety.

### STATEMENT REGARDING FEDERALLY SPONSORED RESEARCH

This invention was made with government support under EB023366 awarded by the National Institutes of Health. The government has certain rights in the invention.

### BACKGROUND

Magnetic resonance imaging (MRI) and spectroscopic imaging (MRSI) are widely used in clinical diagnosis, where the majority of scans relies on detection of tissue water—specifically the proton (¹H) nuclear spin (I=1/2) which has a large gyromagnetic ratio (γ_{H}=42.57 MHz/T). However, to enhance the contrast between normal and diseased tissues, molecular imaging probes are used with multimodal MRI/MRSI platforms. The paramagnetic probe features both exchangeable and non-exchangeable protons, which respectively allow detection schemes for chemical exchange saturation transfer (CEST) and biosensor imaging of redundant deviation in shifts (BIRDS) methods for pH and temperature measurements.

pH and temperature are vital physiological markers of the human body because they are tightly regulated to maintain homeostasis, whereas diseased tissues usually show altered physiological conditions. In cancer tumors, glycolysis is upregulated in relation to oxidative phosphorylation even with sufficient oxygen. Aerobic glycolysis generates excessive amounts of hydrogen ions and lactate, which are extruded into the extracellular milieu, lowering the pH of the tumor microenvironment. Therefore, extracellular acidosis is a hallmark of tumor progression, and precise pH mapping could provide an excellent approach for early detection of cancers.

Similar to pH dysregulation in cancer, electrolyte imbalance also has a role in tumorigenesis. Thus, precise measurement of Na⁺ across different compartments in vivo (interstitial, intracellular and blood) is also considered an important biomarker for diagnosis and prognosis of cancers.

Paramagnetic trivalent lanthanide metal ion (Ln³⁺), e.g., Gd³⁺, Eu³⁺, Yb³⁺, and/or Tm³⁺, complexes are most featured exogenous contrast agents which when added to the blood circulation and upon extravasation into extravascular space enhances tissue water proton relaxation, i.e., shortening both longitudinal (T₁) and transverse (T₂) relaxation times. Region-specific water proton relaxation enhancement improves MRI contrast to delineate lesions with T₁-weighted (positive contrast) and/or T₂-weighted (negative contrast) imaging. Some of these agents also allow measuring pH and temperature through BIRDS, while some of these probes also have CEST properties. However, contrast agents based on lanthanide metal ions can cause toxicity if the ions are released from the ion-chelated complexes.

### BRIEF SUMMARY OF THE INVENTION

In certain aspects, a compound of formula (Ia), formula (Ib), formula (Ic), formula (Id), or formula (Ie), a deuterated analogue of a compound of formula (Ia), formula (Ib), formula (Ic), formula (Id), or formula (Ie), or a pharmaceutically acceptable salt of the preceding compounds is provided.

Compounds of formula (Ia), formula (Ib), formula (Ic), formula (Id), and formula (Ie) have the following structures:

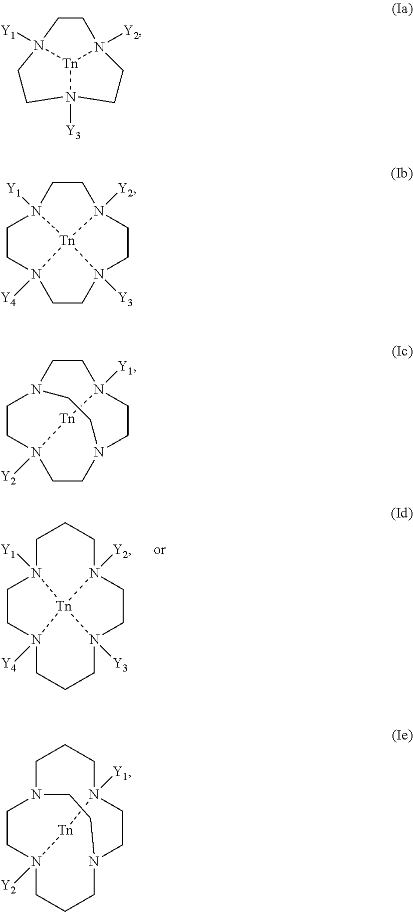

wherein:
each Tn is independently a bivalent (2+) or trivalent (3+) transition metal ion selected from the group consisting of Mn, Fe, Co, Ni, and Zn;
each Y₁, Y₂, Y₃, or Y₄ is independently H or —X—CH₂—P(O)(OH)₂;
each X is independently a bond or LL;
each LL is independently a bivalent, saturated or unsaturated, straight or branched C₁₋₁₂ hydrocarbon chain, wherein 0-6 methylene units of the hydrocarbon are independently replaced with —O—, —S—, —N(R*)—, —OC(═O)—, —C(═O)O—, —S(O)—, —S(O)₂—, —N(R*)S(O)₂—, —S(O)₂N(R*)—, —N(R*)C(═O)—, —C(═O)N(R*)—, —OC(═O)N(R*)—, or —N(R*)C(═O)O—,
   wherein each R* is independently hydrogen, C₁₋₆ alkyl, or C₃₋₆ cycloalkyl;
with the proviso that at least two of Y₁, Y₂, Y₃, and Y₄, if present, are independently —X—CH₂—P(O)(OH)₂.

In certain aspects, compounds of formula (Ia), formula (Ib), formula (Ic), formula (Id), and formula (Ie) are useful as MRI contrast agents.

In certain aspects, a method of imaging a tissue volume using a magnetic resonance imaging (MRI) is provided. In certain embodiments, the method includes contacting a bolus of a contrast agent that includes a compound of formula (Ia), formula (Ib), formula (Ic), formula (Id), or formula (Ie) with the tissue volume. In certain embodiments, the method includes acquiring magnetic resonance image data. In certain embodiments the image data comprises at least one of chemical shift data for non-exchangeable protons in the contrast agent in and surrounding the tissue volume, extracellular sodium concentration surrounding the tissue volume, and extracellular pH data surrounding the tissue volume.

In certain aspects, a method for monitoring the efficacy of a cancer treatment in a patient undergoing chemotherapy is provided. In certain embodiments, the method includes administering a bolus of a contrast agent that includes a compound of formula (Ia), formula (Ib), formula (Ic), formula (Id), or formula (Ie) to the patient. In certain embodiments, the method includes acquiring magnetic resonance image data. In certain embodiments, the image data comprises at least one of chemical shift data for non-exchangeable protons in the contrast agent in and surrounding the tissue volume, extracellular sodium concentration surrounding the tissue volume, and extracellular pH data surrounding the tissue volume.

In certain aspects, a method for monitoring or determining a cancer prognosis in a patient is provided. In certain embodiments, the method includes administering a bolus of a contrast agent that includes a compound of formula (Ia), formula (Ib), formula (Ic), formula (Id), or formula (Ie) to the patient. In certain embodiments, the patient has at least one tissue volume comprising cancerous tissue. In certain embodiments, the method includes acquiring magnetic resonance image data. In certain embodiments, the image data comprises extracellular pH data surrounding the tissue volume.

### BRIEF DESCRIPTION OF THE FIGURES

The drawings illustrate generally, by way of example, but not by way of limitation, various embodiments of the present application.

FIG. 1 shows ligands of interest that form transition metal complexes of the disclosure for application as MR(S)I probes, according to various embodiments. Top row) NOTP⁶⁻ coordinated with Fe²⁺, Co²⁺, Ni²⁺ corresponding complexes [FeNOTP⁴⁻, 6-coordinate; CoNOTP⁴⁻, 6-coordinate; NiNOTP⁴⁻, 6-coordinate]. Middle row) DOTP⁸⁻ coordinated with Fe²⁺, Co²⁺, Ni²⁺ corresponding complexes [FeDOTP⁶⁻, 8-coordinate; CoDOTP⁶⁻, 7-coordinate; NiDOTP⁶⁻, 6-coordinate;]. Bottom row) DOTA-4AMP⁸⁻ coordinated with Fe²⁺, Co²⁺, Ni²⁺ corresponding complexes [FeDOTA-4AMP⁶⁻, 8-coordinate; CoDOTA-4AMP⁶⁻, 7-coordinate; NiDOTA-4AMP⁶⁻, 6-coordinate]. The blue and red protons represent exchangeable and non-exchangeable protons giving rise to CEST and BIRDS detection schemes, respectively.

FIG. 2 is a schematic depiction of MR(S)I probes showing different modalities and applications for pH, temperature, and sodium ion sensing, in accordance with various embodiments.

FIGS. 3A-3C show structures and ¹H NMR spectra of NOTP⁶⁻, DOTP⁸⁻, and DOTA-4AMP⁸⁻ ligands, respectively, in accordance with various embodiments.

FIGS. 4A-4C show ¹H NMR spectra of TnNOTP⁴⁻, TnDOTP⁶⁻, and TnDOTA-4AMP⁶⁻ (Tn²⁺=Fe²⁺, Co²⁺ or Ni²⁺) reveal hyperfine shifted proton peaks. Except the —NH moiety which features exchangeable protons (for CEST), the other labeled peaks are all non-exchangeable protons (for BIRDS). See Table 1 for pH and temperature sensitivities.

FIGS. 5A-5B show representative pH and temperature sensitivities of CoDOTP⁶⁻ and NiDOTP⁶⁻ are shown with MRS proton hyperfine shifted peaks. See Table 1 for pH and temperature sensitivities of TnNOTP⁴⁻, TnDOTP⁶⁻, and TnDOTA-4AMP⁶⁻ (Tn²⁺=Fe²⁺, Co²⁺ or Ni²⁺) complexes.

FIG. 6 shows representative Z-spectra demonstrating the CEST properties of NiDOTA-4AMP⁶⁻.

FIG. 7 shows representative sodium ion shiftability and broadening potentials of TnNOTP⁴⁻ (Tn²⁺=Fe²⁺, Co²⁺ or Ni²⁺). The peak at 0 ppm represents the sodium signal in absence of the paramagnetic agent. See Table 2 for sodium ion shiftability and broadening potentials.

FIG. 8 shows structures of TACN (1,4,7-triazacyclononane), cyclen (1,4,7,10-tetraazacyclododecane), cyclam (1,4,8,11-tetraazacyclotetradecane) and their cross bridged (cb) versions as well as diamsar (1,8-diamino-3,6,10,13,16,19-hexaazabicyclo[6,6,6]-eicosane). In various embodiments, these ligands are suitable for use in the synthesis of compounds of the disclosure and for use in the MRI methods described herein.

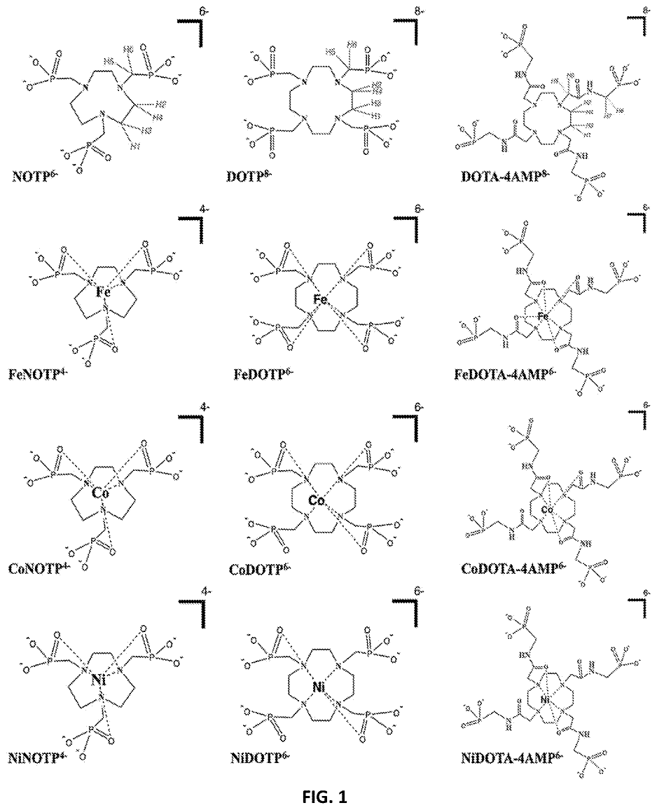

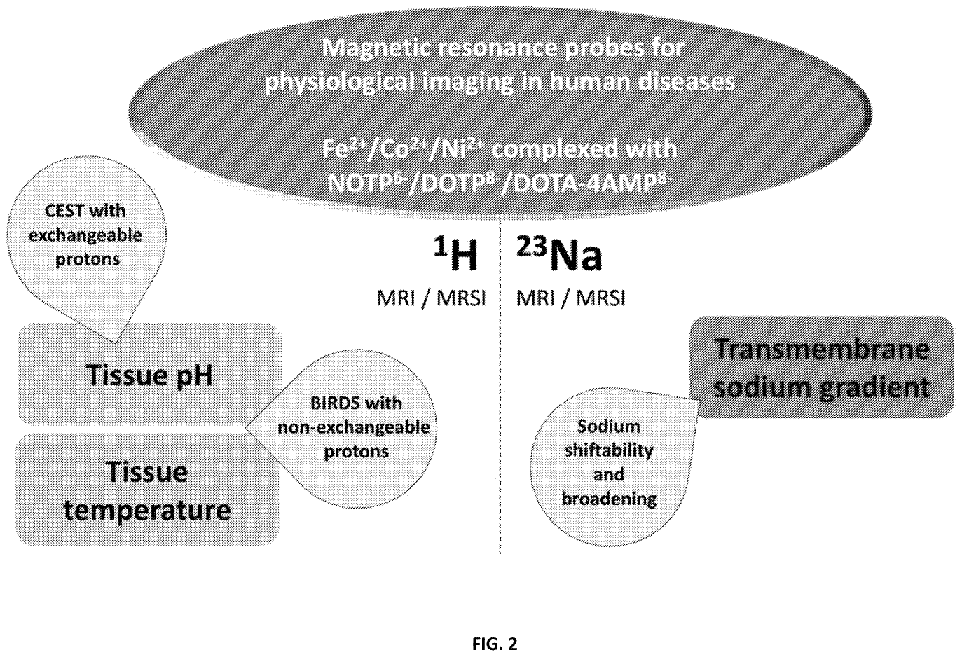

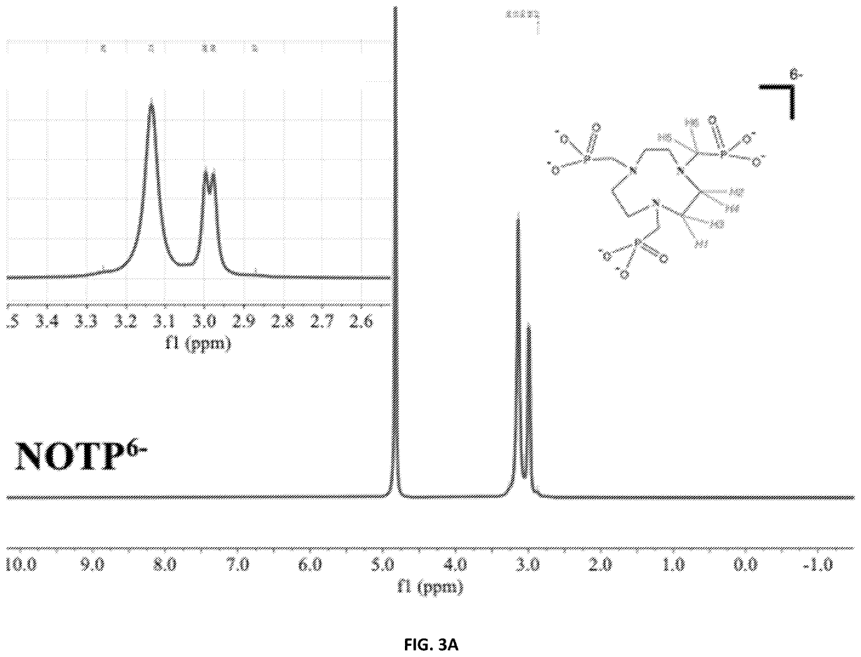

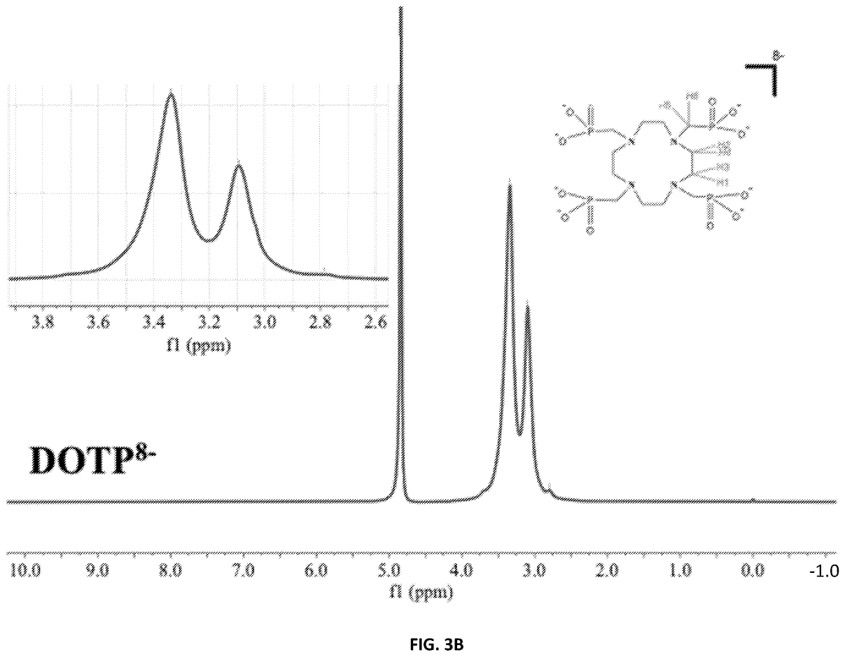

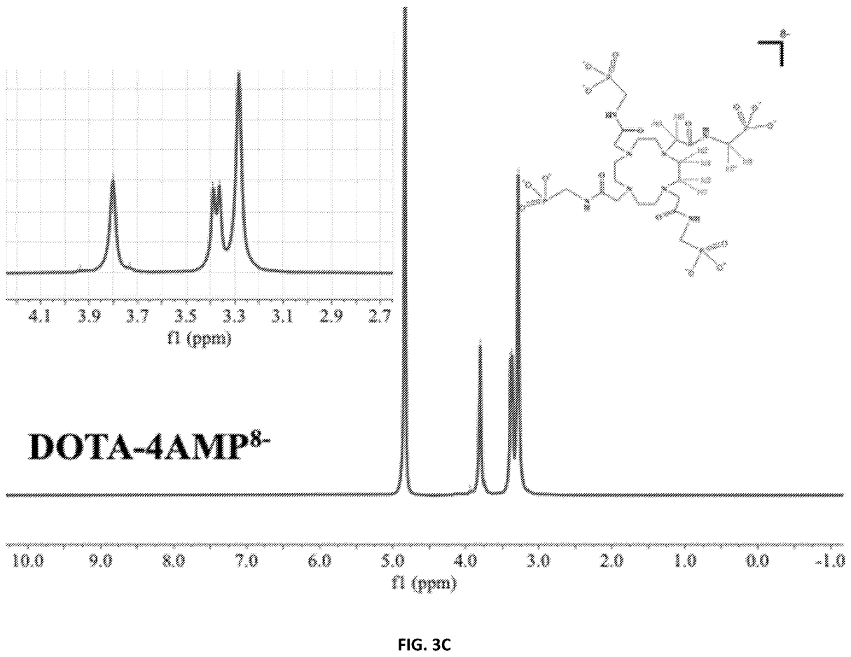

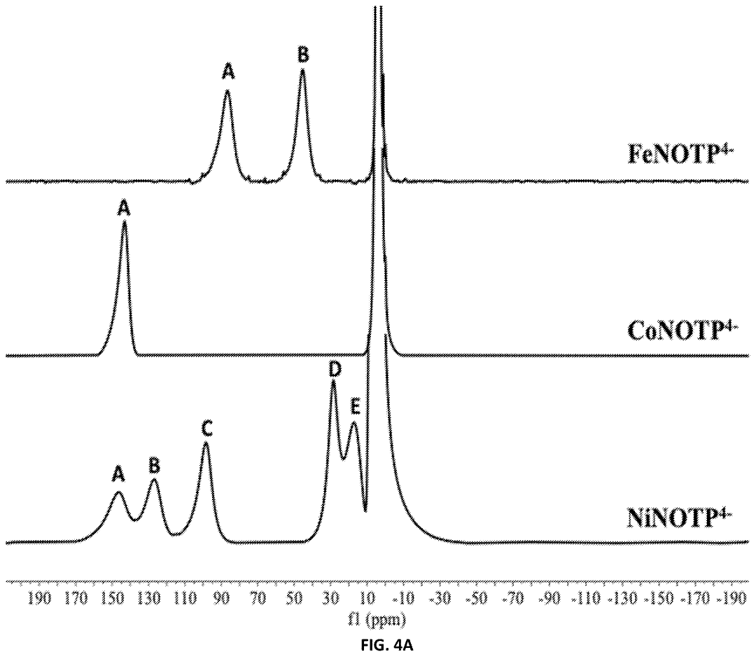

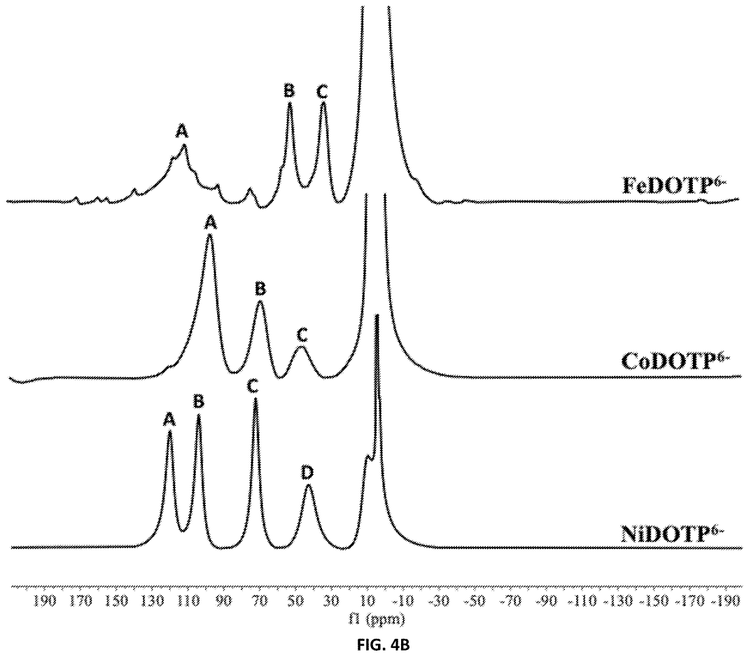

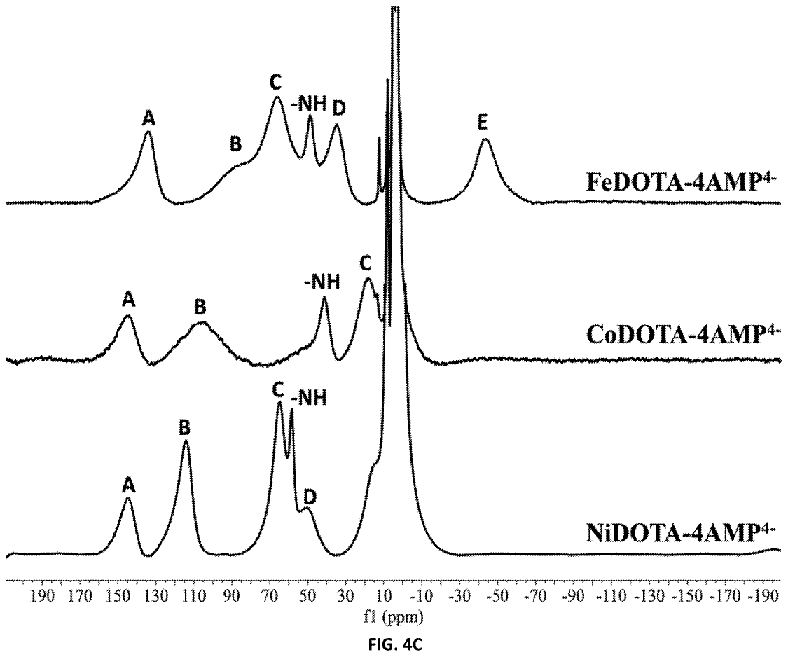

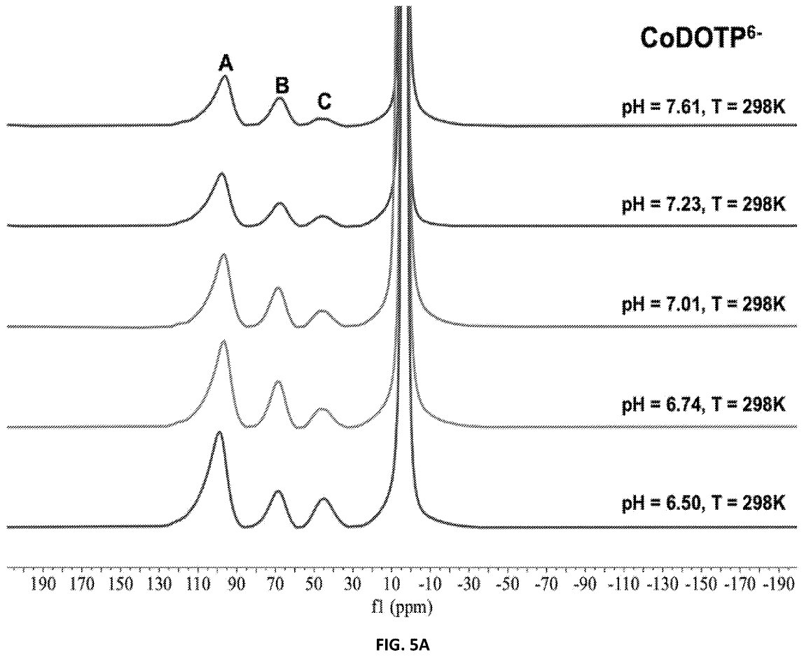

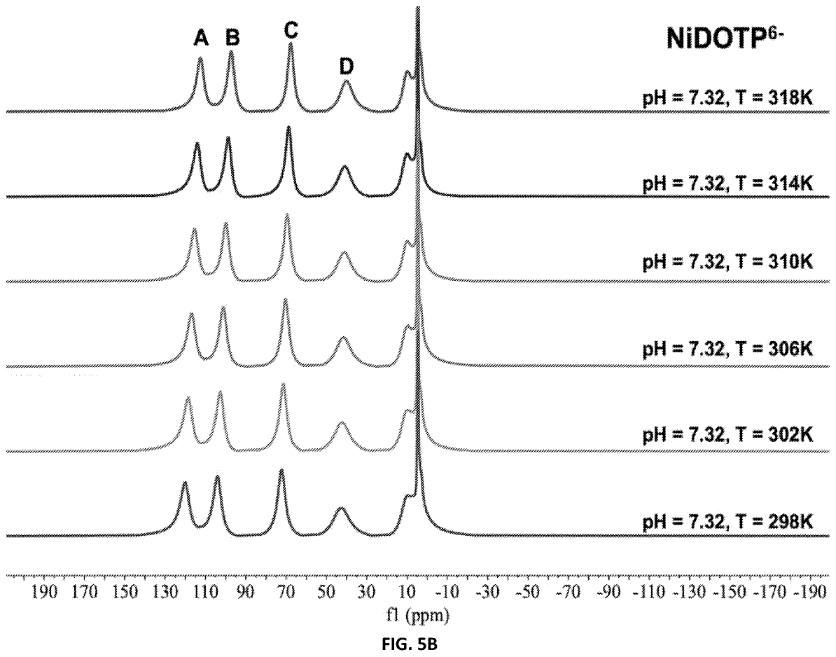

### DETAILED DESCRIPTION

Provided herein are, in one aspect, biocompatible clinically translational divalent transition metal ion (M²⁺) complexed with negatively charged phosphonate macrocyclics (Che⁻ⁿ) to form complexes (MChe⁽⁻ⁿ⁺²⁾). Provided herein are, in one aspect, biocompatible clinically translational trivalent transition metal ion (M³⁺) complexed with negatively charged phosphonate macrocyclics (Che⁻ⁿ) to form complexes (MChe⁽⁻ⁿ⁺³⁾).

In certain embodiments, these complexes are probes for noninvasive measurements of pH, temperature and compartmental sodium ion for early diagnosis and prognosis of cancer tumors. These same transition metal probes can also delineate lesions with T₁-weighted (positive contrast) and/or T₂-weighted (negative contrast) imaging. In certain embodiments, the divalent transition metal complexes described herein are non-toxic and provide clinically important insights into the tumor (and stroke) microenvironment by complementary strengths of ¹H and ²³Na MRSI modalities.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### DEFINITIONS

The term “about” as used herein can allow for a degree of variability in a value or range, for example, within 10%, within 5%, or within 1% of a stated value or of a stated limit of a range and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include —OR, —OOR, —OC(═O)N(R)₂, —CN, CF₃, —OCF₃, R, —C(═O), methylenedioxy, ethylenedioxy, —N(R)₂, —SR, —SOR, SO₂R, SO₂N(R)₂, SO₃R, —C(═O)R, —C(═O)C(═O)R, —C(═O)CH₂C(═O)R, —C(═S)R, C(═O)OR, —OC(═O)R, —C(═O)N(R)₂, —OC(═O)N(R)₂, —C(═S)N(R)₂, (CH₂)₀₋₂N(R)C(═O)R, (CH₂)₀₋₂N(R)N(R)₂, N(R)N(R)C(═O)R, N(R)N(R)C(═O)OR, N(R)N(R)CON(R)₂, N(R)SO₂R, N(R)SO₂N(R)₂, N(R)C(═O)OR, N(R)C(═O)R, N(R)C(S)R, N(R)C(═O)N(R)₂, N(R)C(S)N(R)₂, N(COR)COR, N(OR)R, —C(═NH)N(R)₂, —C(═O)N(OR)R, —C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The substitution can be direct substitution, whereby the hydrogen atom is replaced by a functional group or substituent, or an indirect substitution, whereby an intervening linker group replaces the hydrogen atom, and the substituent or functional group is bonded to the intervening linker group. A non-limiting example of direct substitution is: RR—H→RR—Cl, wherein RR is an organic moiety/fragment/molecule. A non-limiting example of indirect substitution is: RR—H→RR-(LL)_{zz}-Cl, wherein RR is an organic moiety/fragment/molecule, LL is an intervening linker group, and ‘zz’ is an integer from 0 to 100 inclusive. When zz is 0, LL is absent, and direct substitution results. The intervening linker group LL is at each occurrence independently selected from the group consisting of —H, —O—, —OR, —S—, —S(═O)—, —S(═O)₂—, —SR, —N(R)—, —NR₂, —CR≡, —C≡, —CH₂—, —CHR—, —CR₂—, —CH₃, —C(═O)—, —C(═NR)—, and combinations thereof. (LL)_{zz} can be linear, branched, cyclic, acyclic, and combinations thereof.

The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, —OR, —OC(═O)N(R)₂, —CN, NO, NO₂, ONO₂, azido, CF₃, OCF₃, —R, —O— (oxo), S (thiono), C(═O), S(═O), methylenedioxy, ethylenedioxy, N(R)₂, SR, SOR, SO₂R, SO₂N(R)₂, SO₃R, —C(═O)R, —C(═O)C(═O)R, —C(═O)CH₂C(═O)R, —C(═S)R, —C(═O)OR, —OC(═O)R, —C(═O)N(R)₂, —OC(═O)N(R)₂, —C(═S)N(R)₂, (CH₂)₀₋₂N(R)C(═O)R, (CH₂)₀₋₂N(R)N(R)₂, N(R)N(R)C(═O)R, N(R)N(R)C(═O)OR, N(R)N(R)CON(R)₂, N(R)SO₂R, N(R)SO₂N(R)₂, N(R)C(═O)OR, N(R)C(═O)R, N(R)C(S)R, N(R)C(═O)N(R)₂, N(R)C(S)N(R)₂, N(COR)COR, N(OR)R, —C(═NH)N(R)₂, —C(═O)N(OR)R, and —C(═NOR)R, wherein R can be hydrogen or a carbon-based moiety; for example, R can be hydrogen. (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)=CH₂, —C(CH₃)═CH(CH₃), —C(CH₂CH₃)=CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “heterocycloalkyl” as used herein refers to a cycloalkyl group as defined herein in which one or more carbon atoms in the ring are replaced by a heteroatom such as O, N, S, P, and the like, each of which may be substituted as described herein if an open valence is present, and each may be in any suitable stable oxidation state.

The term “aryl” as used herein refers to cyclic aromatic hydrocarbon groups that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. The term heterocyclyl includes rings where a CH₂ group in the ring is replaced by one or more C═O groups, such as found in cyclic ketones, lactones, and lactams. Examples of heterocyclyl groups containing a C═O group include, but are not limited to, β-propiolactam, γ-butyrolactam. δ-valerolactam, and ε-caprolactam, as well as the corresponding lactones. A heterocyclyl group designated as a C₂-heterocyclyl can be a 5-ring with two carbon atoms and three heteroatoms, a 6-ring with two carbon atoms and four heteroatoms and so forth. Likewise, a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be substituted or unsubstituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise, a C₄-heteroaryl can be a 5-ring with one heteroatom, a 6-ring with two heteroatoms, and so forth. The number of carbon atoms plus the number of heteroatoms sums up to equal the total number of ring atoms. A heterocyclyl ring designated C_{x-y} can be any ring containing ‘x’ members up to ‘y’ members, including all intermediate integers between ‘x’ and ‘y’ and that contains one or more heteroatoms, as defined herein. In a ring designated C_{x-y}, all non-heteroatom members are carbon. Heterocyclyl rings designated C_{x-y} can also be polycyclic ring systems, such as bicyclic or tricyclic ring systems. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be substituted or unsubstituted with groups as discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxycyclohexyl)ethyl, 2-(2,3-epoxycylopentyl)ethyl, 2-(4-methyl-3,4-epoxycyclohexyl)propyl, 2-(3,4-epoxy-3-methylcylohexyl)-2-methylethyl, and 5,6-epoxyhexyl.

The term “monovalent” as used herein refers to a substituent connecting via a single bond to a substituted molecule. When a substituent is monovalent, such as, for example, F or Cl, it is bonded to the atom it is substituting by a single bond.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “solvent” as used herein refers to a liquid that can dissolve a solid, liquid, or gas. Non-limiting examples of solvents are silicones, organic compounds, water, alcohols, ionic liquids, and supercritical fluids.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “room temperature” as used herein refers to a temperature of about 15 °C to 28 °C.

The term “standard temperature and pressure” as used herein refers to 20 °C and 101 kPa.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, a “diagnostically effective amount” or “pharmaceutically effective amount” is an amount sufficient to achieve or provide a clinically relevant MRI image or images of a tissue volume, such as a cancer, without causing any adverse reaction in the subject to which the compounds is administered. In some embodiments, a clinically relevant image is one that a physician can use to base some or all of a diagnosis.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases and/or buffers, including inorganic acids or bases, organic acids, or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985. Easton, PA), which is incorporated herein by reference.

The terms “patient,” “subject.” or “individual” are used interchangeably herein, and refer to any animal, or cells thereof whether in vitro or in situ, amenable to the methods described herein. In a non-limiting embodiment, the patient, subject or individual is a human.

### Preparation of Compounds

Compounds of formula (Ia), (Ib), (Ic), (Id), or (Ie) or otherwise described herein can be prepared by the general schemes described herein, using the synthetic method known by those skilled in the art. The following examples illustrate non-limiting embodiments of the compound(s) described herein and their preparation.

In various embodiments, a method of making a compound, or a pharmaceutically acceptable salt thereof of formula (Ia), (Ib), (Ic), (Id), or (Ie) is provided.

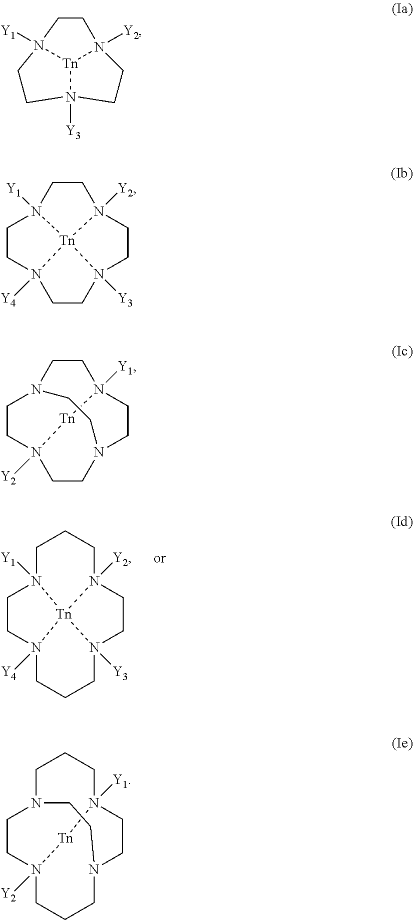

In certain embodiments, each Tn is independently a divalent or trivalent transition metal ion selected from the group consisting of Mn, Fe, Co, Ni, and Zn.

In certain embodiments, each Y₁, Y₂, Y₃, or Y₄ is independently H or —X—CH₂—P(O)(OH)₂.

In certain embodiments, each X is independently a bond or LL.

In certain embodiments, each LL is independently a bivalent, saturated or unsaturated, straight or branched C₁₋₁₂ hydrocarbon chain, wherein 0-6 methylene units of the hydrocarbon are independently replaced with —O—, —S—, —N(R*)—, —OC(═O)—, —C(═O)O—, —S(O)—, —S(O)₂—, —N(R*)S(O)₂—, —S(O)₂N(R*)—, —N(R*)C(═O)—, —C(═O)N(R*)—, —OC(═O)N(R*)—, or —N(R*)C(═O)O—.

In certain embodiments, each R* is independently hydrogen, C₁₋₆ alkyl, or C₃₋₆ cycloalkyl.

In certain embodiments, at least two of Y₁, Y₂, Y₃, and Y₄, if present, are independently —X—CH₂—P(O)(OH)₂.

In certain embodiments, X is a bond.

In certain embodiments, X is —CH₂—.

In certain embodiments, X is —CH₂—CH₂—.

In certain embodiments, X is —CH₂—C(═O)—NH—.

In certain embodiments, Tn is Mn (II) or Mn(III) in the compound formula (Ia), (Ib), (Ic), (Id), or (Ie).

In certain embodiments, Tn is Fe (II) or Fe(III) in the compound formula (Ia), (Ib), (Ic), (Id), or (Ie).

In certain embodiments, Tn is Co (II) or Co(III) in the compound formula (Ia), (Ib), (Ic), (Id), or (Ie).

In certain embodiments, Tn is Ni (II) or Ni(III) in the compound formula (Ia), (Ib), (Ic), (Id), or (Ie).

In certain embodiments, Tn is Zn (II) in the compound formula (Ia), (Ib), (Ic), (Id), or (Ie).

In certain embodiments, LL does not comprise a —O—O— bond. In certain embodiments, LL does not comprise a —S—S— bond. In certain embodiments, LL does not comprise a —N—N— bond. In certain embodiments, LL does not comprise a —N—O— bond.

In certain embodiments, the method comprises contacting a salt of Tn with a ligand, or a salt thereof, having the structure

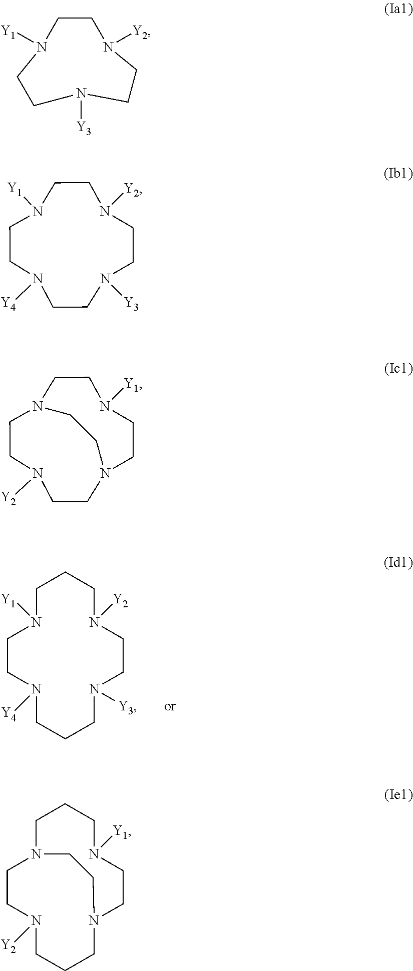

in an organic solvent under inert atmosphere to produce the compound of formula (Ia), (Ib), (Ic), (Id), or (Ie), respectively.

In various embodiments, the solvent is an alcohol, such as but not limited to methanol, ethanol, and the like.

In various embodiments, the ligand is an alkali metal salt or alkaline earth metal salt. In various embodiments, the ligand is unsalted (does not have a counterion associated with it) and is in the form of a free based and phosphonic acid/phosphonate. In various embodiments, the salt of Tn is a fluoride, chloride, bromide, or nitrate salt.

As discussed herein, compounds of the disclosure are prepared, in some embodiments, in an organic solvent under inert atmosphere, so that divalent transition metal ions such as Mn²⁺, Fe²⁺, Co²⁺ or Ni²⁺, or trivalent transition metal ions, cannot be oxidized. Ligands such as NOTP⁶⁻, DOTP⁸⁻, or DOTA-4AMP⁸⁻ are dissolved in ethanol (absolute) and purged with inert gas (nitrogen gas). Ligands may be used in unsalted form (free amine/phosphonic acids or phosphonate) or as alkali metal salts or alkaline earth metal salts, such as Li, Na, K, Mg, Ca, and the like.

In various embodiments, the ligands have the following structures:

| Ligand | Chemical Name |
|---|---|
| 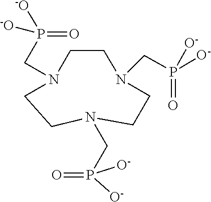 | 1,4,7-Triazacyclo- nonane-1,4,7- tri(methylene phosphonate) |
| (NOTP⁶⁻) | |
| 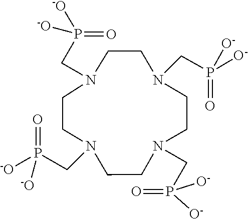 | 1,4,7,10- Tetraazacyclo- dodecane-1,4,7,10- tetra(methylene phosphonate) |
| (DOTP⁸⁻) | |
| 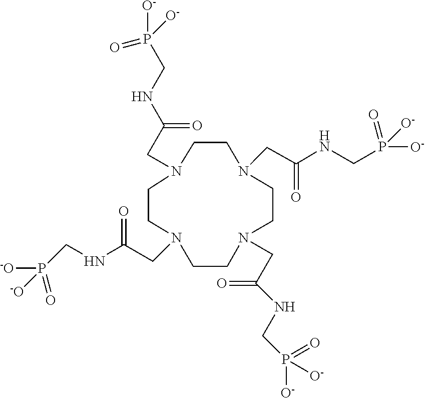 | 1,4,7,10- tetraazacyclo- dodecane-1,4,7,10- tetrakis(acetamido- methylene phosphonate) |
| (DOTA-4AMP⁸⁻) | |

In various embodiments, the transition metal salts (specific to each different Tn²⁺ or Tn³⁺ ion) are dissolved in ethanol (absolute) under inert atmosphere of nitrogen gas. Suitable metal salts include, but are not limited to, MnCl₂, MnBr₂, MnSO₄, Mn(NO₃)₂, MnCl₃, MnBr₃, Mn₂(SO₄)₃, Mn(NO₃)₃, FeCl₂, FeBr₂, FeSO₄, Fe(NO₃)₂, FeCl₃, FeBr₃, Fe₂(SO₄)₃, Fe(NO₃)₃, CoCl₂, CoBr₂, Co(NO₃)₂, CoSO₄, CoCl₃, CoBr₃, Co(NO₃)₃, C₀₂(SO₄)₃, NiCl₂, NiBr₂, Ni(NO₃)₂, NiSO₄, NiCl₃, NiBr₃, Ni(NO₃)₃, Ni₂(SO₄)₃, ZnCl₂, ZnBr₂, Zn(NO₃)₂, ZnSO₄, and the like. Also suitable are hydrates of these transition metal salts, and other salts that are soluble in the organic solvent in which the complexation with the ligand is performed. The respective ligand (NOTP⁶⁻, DOTP⁸⁻, or DOTA-4AMP⁸⁻) solution is mixed with the respective transition metal salt solution and stirred under inert atmosphere for 24 h. After completion of the synthesis, the complex is purified through set of physiochemical methods and chromatography. The pH and temperature sensing abilities of compounds of the disclosure are set forth in Table 1. The sodium sensing abilities of compounds of the disclosure are set forth in Table 2.

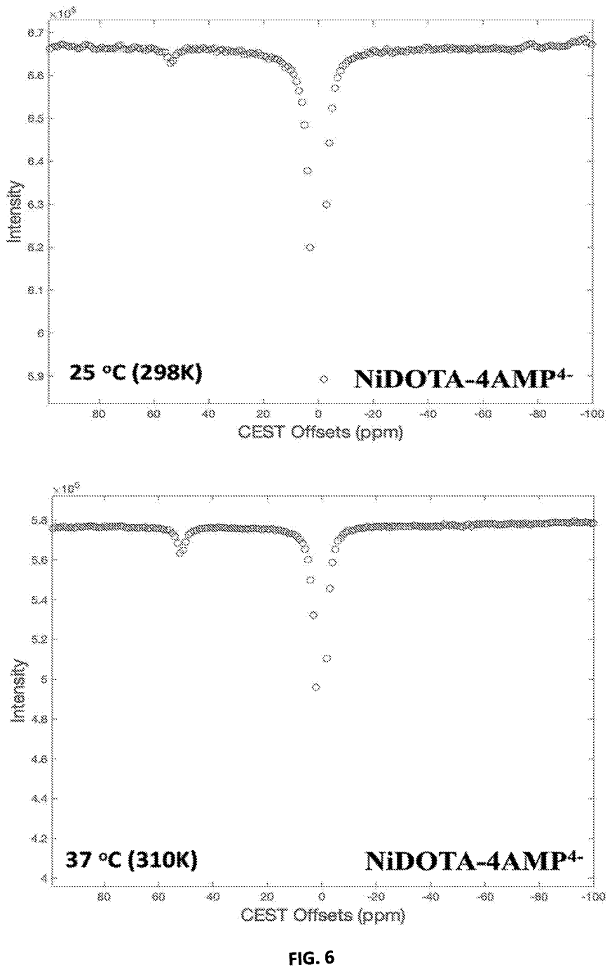

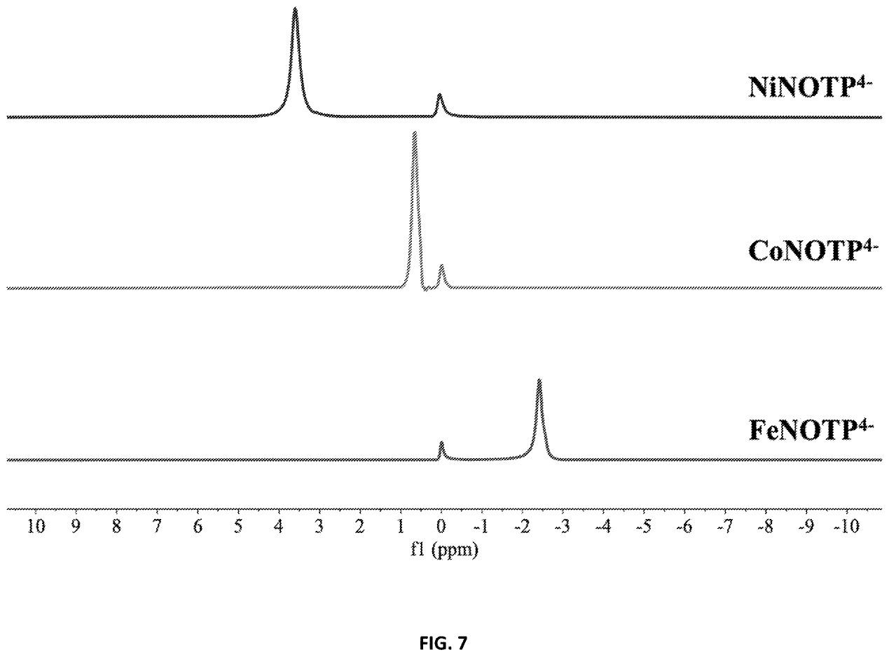

TABLE 1
pH and temperature sensing abilities of
TnNOTP⁴⁻, TnDOTP⁶⁻, TnDOTA-4AMP⁶⁻
(Tn²⁺ = Fe²⁺, Co²⁺ or Ni²⁺) compared
with equivalent complexes of trivalent
thulium ion (Tm³⁺). While all probes are
BIRDS sensitive, note the DOTA-4AMP⁸⁻
complexes are also CEST active (see Figure 6).

| | Non- | | pH |
|---|---|---|---|
| | exchangeable | T (ppm/ | (ppm/ |
| Probes | protons | ° C.) | pH unit) |
| TmNOTP³⁻ | A (@56) | 0.351 | -2.58 |
| | B (@23) | 0.140 | -0.56 |
| | C (@-20) | -0.250 | 1.88 |
| TmDOTP⁵⁻ | H2 | -0.6 | 4.2 |
| [Coman D., et al., NMR | H3 | -0.5 | 3.6 |
| Biomed. 2010; 23, 277-285] | H6 | 1.1 | -3.9 |
| TmDOTA-4AMP⁵⁻ | H1 | 0.54 | 1.8 |
| [Huang Y., et al., Contrast | H2 | -0.24 | -0.63 |
| Media Mol. Imaging 2015, | H3 | -0.188 | -0.25 |
| 10, 51-58] | H4 | -1.49 | -3.9 |
| | H6 | 0.45 | 1.2 |
| | H8 | 0.170 | -4.0 |
| FeNOTP⁴⁻ | A | 0.240 | -- |
| | B | 0.149 | -- |
| CONOTP⁴⁻ | A | 0.948 | 6.20 |
| NINOTP⁴⁻ | A | 0.516 | 1.35 |
| | B | 0.468 | 1.54 |
| | C | 0.342 | -- |
| | D | 0.066 | -- |
| | E | 0.061 | 1.63 |
| FeDOTP⁶⁻ | A | 0.345 | -- |
| | B | 0.130 | -- |
| | C | 0.045 | -- |
| CODOTP⁶⁻ | A | 0.434 | 6.0 |
| | B | 0.234 | 1.42 |
| | C | 0.186 | -- |
| NiDOTP⁶⁻ | A | 0.383 | 1.10 |
| | B | 0.342 | -0.22 |
| | C | 0.168 | -0.26 |
| | D | 0.108 | 0.50 |
| FeDOTA-4AMP⁶⁻ | A | 0.413 | -- |
| | C | -- | -- |
| | D | -- | -- |
| | E | -0.306 | -- |
| | -NH | 0.158 | -- |
| CoDOTA-4AMP⁶⁻ | A | 0.576 | -- |
| | B | -- | -- |
| | -NH | 0.161 | -- |
| NiDOTA-4AMP⁶⁻ | A | 0.520 | -- |
| | B | 0.410 | 0.75 |
| | C | 0.226 | 0.63 |
| | -NH | 0.184 | -- |

TABLE 2
Sodium sensing abilities of TnNOTP⁴⁻, TnDOTP⁶⁻, TnDOTA-4AMP⁶⁻
(Tn²⁺ = Fe²⁺, Co²⁺ or Ni²⁺) compared with
reported shiftability and broadening potentials. Note that
negative and positive shiftability values indicate downfield and
upfield signals from unshifted sodium signal.

| | Shiftability (S_{c}) | Broadening (β_{c}) |
|---|---|---|
| Sensor | (ppm/mM) | (ppm/mM) |
| TmNOTP³⁻ | -0.568 | 0.018 |
| TmDOTP⁵⁻ | -2.03 | 0.042 |
| TmDOTA-4AMP⁵⁻ | -- | -- |
| GdNOTP³⁻ | -0.0435 | 0.0117 |
| GdDOTP⁵⁻ | -0.040 | 0.036 |
| GdDOTA-4AMP⁵⁻ | -- | -- |
| FeNOTP⁴⁻ | +0.318 | 0.015 |
| CONOTP⁴⁻ | +0.140 | 0.013 |
| NINOTP⁴⁻ | -0.525 | 0.011 |
| FeDOTP⁶⁻ | +0.688 | 0.023 |
| CODOTP⁶⁻ | -0.207 | 0.016 |
| NiDOTP⁶⁻ | -0.144 | 0.012 |
| FeDOTA-4AMP⁶⁻ | -0.477 | 0.020 |
| CoDOTA-4AMP⁶⁻ | -0.172 | 0.022 |
| NiDOTA-4AMP⁶⁻ | +0.136 | 0.018 |

In various embodiments, a compound of formula (Ia), (Ib), (Ic), (Id), or (Ie), a deuterated analog thereof, or a pharmaceutically acceptable salt thereof, is provided:

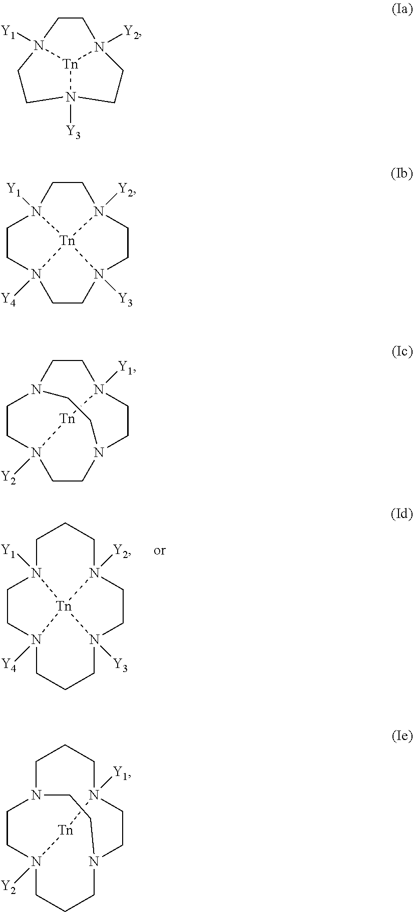

In certain embodiments of the compound of formula (Ia), (Ib), (Ic), (Id), or (Ie), each Tn is independently a transition metal selected from the group consisting of Mn, Fe, Co, Ni, and Zn.

In certain embodiments of the compound of formula (Ia), (Ib), (Ic), (Id), or (Ie), each Y₁, Y₂, Y₃, or Y₄ is independently H or —X—CH₂—P(O)(OH)₂.

In certain embodiments of the compound of formula (Ia), (Ib), (Ic), (Id), or (Ie), each X is independently a bond or LL.

In certain embodiments of the compound of formula (Ia), (Ib), (Ic), (Id), or (Ie), each LL is independently a bivalent, saturated or unsaturated, straight or branched C₁₋₁₂ hydrocarbon chain, wherein 0-6 methylene units of the hydrocarbon are independently replaced with —O—, —S—, —N(R*)—, —OC(═O)—, —C(═O)O—, —S(O)—, —S(O)₂—, —N(R*)S(O)₂—, —S(O)₂N(R*)—, —N(R*)C(═O)—, —C(═O)N(R*)—, —OC(═O)N(R*)—, or —N(R*)C(═O)O—.

In certain embodiments of the compound of formula (Ia), (Ib), (Ic), (Id), or (Ie), each R* is independently hydrogen, C₁₋₆ alkyl, or C₃₋₆ cycloalkyl.

In certain embodiments of the compound of formula (Ia), (Ib), (Ic), (Id), or (Ie), at least two of Y₁, Y₂, Y₃, and Y₄, if present, are independently —X—CH₂—P(O)(OH)₂.

In certain embodiments, LL does not comprise a —O—O— bond. In certain embodiments, LL does not comprise a —S—S— bond. In certain embodiments, LL does not comprise a —N—N-bond. In certain embodiments, LL does not comprise a —N—O— bond.

By “deuterated analog” of a compound of formula (Ia), (Ib), (Ic), (Id), or (Ie) it is meant that one or more of the non-exchangeable hydrogen atoms (protons) in the compound of formula (Ia), (Ib), (Ic), (Id), or (Ie) is/are replaced by deuterium. For example, and without limitation,

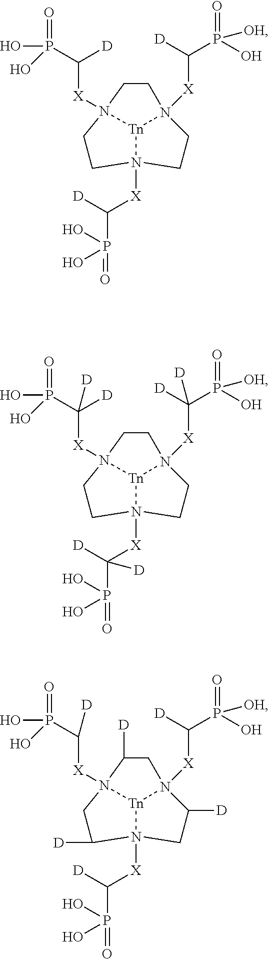

and the like. In certain embodiments, the compound of formula (Ia), (Ib), (Ic), (Id), or (Ie) is perdeuterated such that all non-exchangeable hydrogen atoms (protons) in the compound are deuterium atoms.

Contemplated herein is every deuterated isomer of any particular compound of formula (Ia), (Ib), (Ic), (Id), or (Ie) as if each was individually drawn out. Contemplated herein is every deuterated isomer of a compound selected from the group consisting of

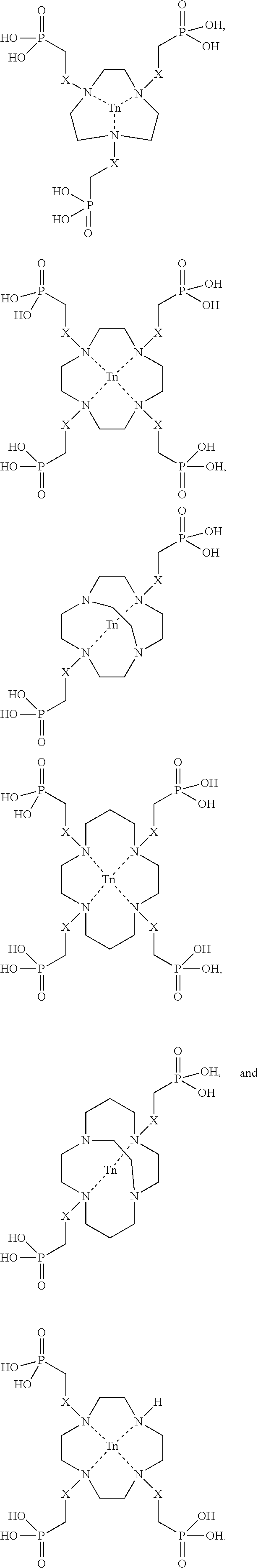

In various embodiments, the compound of the disclosure is a transition metal complex of a derivative of the ligand diamsar:

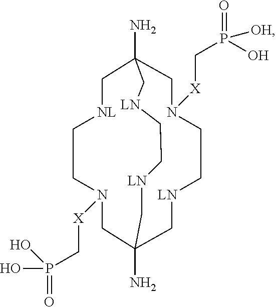

wherein each L is independently H or —X—CH₂—P(═O)(OH)₂, wherein X is as defined herein.

In various embodiments, X is

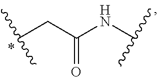 [SMILES: *CC(=O)N*]

and
wherein the * designates the point of attachment to the ring N atom in formula (Ia), (Ib), (Ic), (Id), or (Ie).

In various embodiments. Tn is in the +2 oxidation state. In various embodiments. Tn is Fe, Co. or Ni.

In various embodiments, Tn is in the +3 oxidation state. In various embodiments, Tn is Fe, Co, or Ni.

In various embodiments, the compound of formula (Ia), (Ib), (Ic), (Id), or (Ie) has the structure:

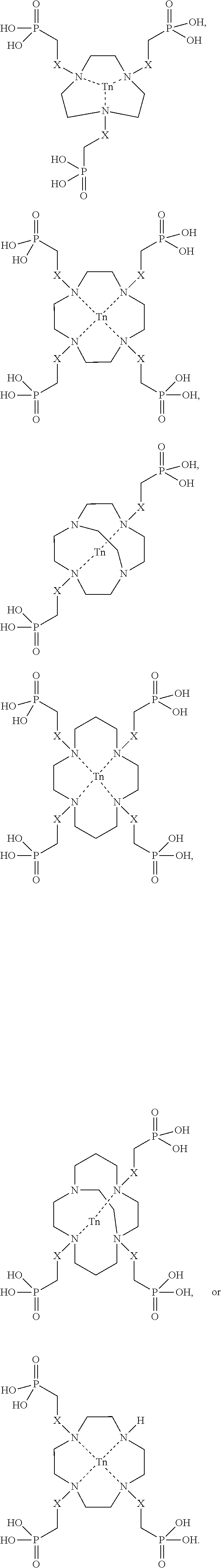

In various embodiments, the compound of formula (Ia), (Ib), (Ic), (Id), or (Ie) has the structure:

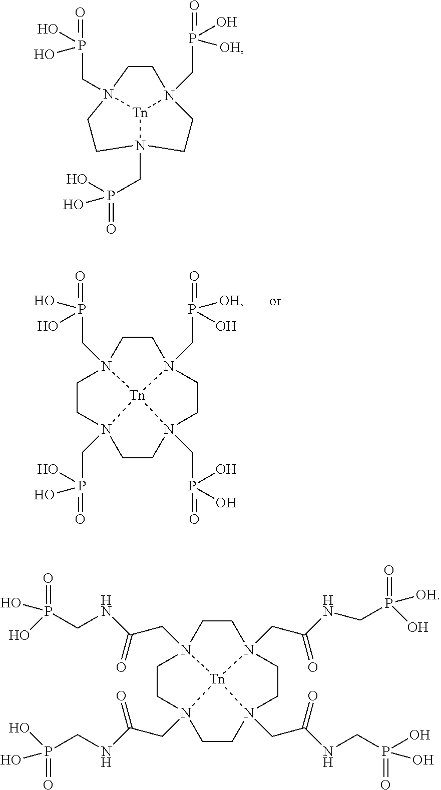

In various embodiments, pharmaceutical composition including a compound of formula (Ia), (Ib), (Ic), (Id), or (Ie) and at least one pharmaceutically acceptable carrier or excipient is provided. In various embodiments, the pharmaceutical composition is an aqueous solution of the compound of formula (Ia), (Ib), (Ic), (Id), or (Ie) and water or isotonic saline solution.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solution forms with pharmaceutically acceptable solvents such as water, and ethanol. In other embodiments, the compounds described herein exist in powdered form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically active form of the compound. In other embodiments, a prodrug is enzymatically metabolized by one or more steps or processes to the biologically, pharmaceutically active form of the compound.

In certain embodiments, sites on, for example, the aromatic ring portion of compound(s) described herein are susceptible to various metabolic reactions. Incorporation of appropriate substituents on the aromatic ring structures may reduce, minimize, or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotope-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S.

In certain embodiments, isotope-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotope-labeled compounds are prepared by any suitable method or by processes using an appropriate isotope-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

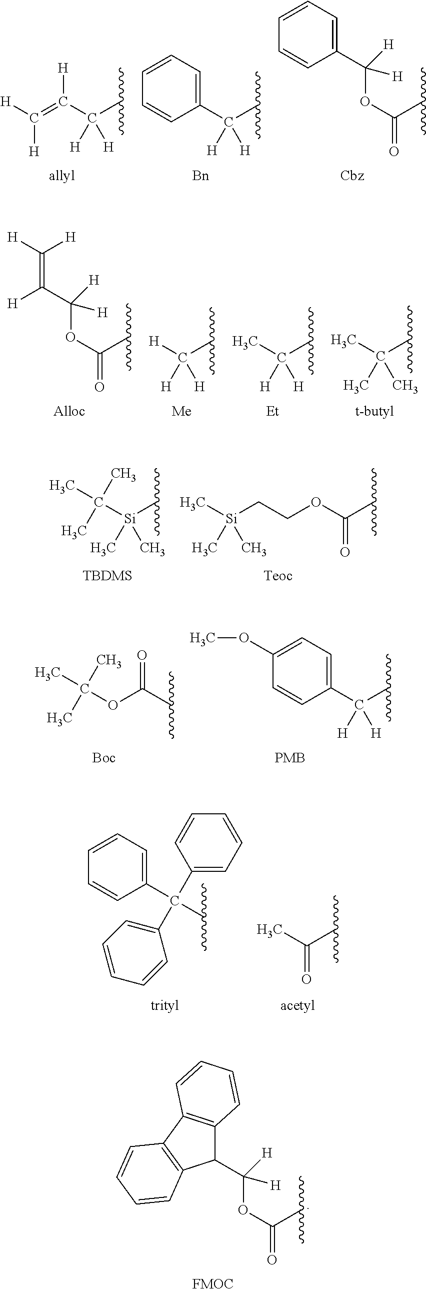

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Pharmacology

In various embodiments, the compound(s) described herein can be administered to a subject in an amount ranging from about 0.01 mg/kg to about 200 mg/kg, or about 0.5 mg/kg to about 190 mg/kg, or about 0.75 mg/kg to about 180 mg/kg, or about 1 mg/kg to about 170 mg/kg, or about 1.5 mg/kg to about 160 mg/kg, or about 2 mg/kg to about 150 mg/kg, or about 2.5 mg/kg to about 140 mg/kg, or about 3 mg/kg to about 130 mg/kg, or about 3.5 mg/kg to about 120 mg/kg, or about 4 mg/kg to about 110 mg/kg, or about 4.5 mg/kg to about 100 mg/kg, or about 5 mg/kg to about 95 mg/kg, or about 5.5 mg/kg to about 90 mg/kg, or about 6 mg/kg to about 85 mg/kg, or about 6.5 mg/kg to about 80 mg/kg, or about 7 mg/kg to about 75 mg/kg, or about 7.5 mg/kg to about 70 mg/kg, or about 8 mg/kg to about 65 mg/kg, or about 8.5 mg/kg to about 60 mg/kg, or about 9 mg/kg to about 55 mg/kg or about 9.5 mg/kg to about 50 mg/kg, or about 10 mg/kg to about 45 mg/kg.

In various embodiments, the compound(s) described herein can be administered to a subject in an amount that is less than, equal to, or greater than about 0.01 mg/kg, 0.05 mg/kg, 0.1 mg/kg, 0.25 mg/kg, 0.5 mg/kg, 0.75 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 12 mg/kg, 14 mg/kg, 16 mg/kg, 18 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 100 mg/kg, 105 mg/kg, 110 mg/kg, 115 mg/kg, 120 mg/kg, 125 mg/kg, 130 mg/kg, 140 mg/kg, 145 mg/kg, 150 mg/kg, 155 mg/kg, 160 mg/kg, 170 mg/kg, 175 mg/kg, 180 mg/kg, 185 mg/kg, 190 mg/kg, 195 mg/kg, or 200 mg/kg.

### Compositions

The compositions containing the compound(s) described herein include a pharmaceutical composition comprising at least one compound as described herein and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

Methods of MRI Imaging with Compounds of the Disclosure

In various embodiments, the disclosure provides a method of imaging a tissue volume using magnetic resonance imaging (MRI) and a compound of the disclosure, such as a compound of formula (Ia), (Ib), (Ic), (Id), or (Ie).

In certain embodiments, the method includes contacting a bolus of a contrast agent comprising the compound of the disclosure with the tissue volume.

In certain embodiments, the method includes acquiring magnetic resonance image data.

In certain embodiments, the image data comprises at least one of chemical shift data for non-exchangeable protons in the contrast agent in and surrounding the tissue volume, extracellular sodium concentration surrounding the tissue volume, and extracellular pH data surrounding the tissue volume.

As used herein, “surrounding the tissue volume” means the volume of extracellular fluid adjacent to a particular tissue volume of interest, such as a cancerous tumor.

In various embodiments, a method for monitoring the efficacy of a cancer treatment in a patient undergoing chemotherapy is provided.

In certain embodiments, the method includes administering a bolus of a contrast agent comprising the compound of the disclosure, such as a compound of formula (Ia), (Ib), (Ic), (Id), or (Ie) to the patient.

In certain embodiments, the method includes acquiring magnetic resonance image data. In certain embodiments, the image data comprises at least one of chemical shift data for non-exchangeable protons in the contrast agent in and surrounding the tissue volume, extracellular sodium concentration surrounding the tissue volume, and extracellular pH data surrounding the tissue volume.

In various embodiments, a method for monitoring or determining a cancer prognosis in a patient is provided.

In certain embodiments, the method includes administering a bolus of a contrast agent comprising the compound of the disclosure, such as a compound of formula (Ia), (Ib), (Ic), (Id), or (Ie) to the patient.

In certain embodiments, the patient has at least one tissue volume comprising cancerous tissue.

In certain embodiments, the method includes acquiring magnetic resonance image data. In certain embodiments, the image data comprises extracellular pH data surrounding the tissue volume.

In various embodiments, a greater volume of measured acidic extracellular pH in an environment surrounding the tissue volume is correlated with a more aggressive cancer. In various embodiments whether a volume is acidic is determined relative to a volume in the same sample or individual that is known not to have any cancerous tumors (normal tissue).

In various embodiments, the tissue volume includes or is a cancerous tumor.

In various embodiments, acquiring magnetic resonance image data includes measuring of the chemical exchange saturation transfer (CEST) of NH protons in the contrast agent and mapping of extracellular pH of the cancerous tumor.

In various embodiments, acquiring magnetic resonance data includes a) exposing the contrast agent to a saturation radiofrequency (RF) pulse. In various embodiments, acquiring magnetic resonance data includes b) exposing the contrast agent to at least one sampling RF pulse. In various embodiments, acquiring magnetic resonance data includes c) exposing the contrast agent to one or more RF pulses to obtain CEST data.

In various embodiments, the RF pulse has an amplitude of about 0.1 to about 50 μT,

In various embodiments, the tissue volume includes an in vitro tissue sample. In various embodiments, the tissue volume includes in vivo tissue in a mammal. In various embodiments, the mammal is a human.

In various embodiments, compounds of the disclosure such as TnNOTP⁴⁻, TnDOTP⁶⁻, TnDOTA-4AMP⁶⁻ agents allow for delineation of lesions with T₁-weighted (positive contrast) and/or T₂-weighted (negative contrast) imaging.

In various embodiments, compounds of the disclosure such as TnNOTP⁴⁻, TnDOTP⁶⁻, TnDOTA-4AMP⁶⁻ are agents for pH and temperature sensing ability through a MRSI hyperfine shift method (Table 1).

In various embodiments, measuring of the chemical shift of one or more protons in the compound includes the mapping of extracellular pH of a cancer tumor.

In various embodiments, compounds of the disclosure such as TnDOTA-4AMP⁶⁻ are agents for pH and temperature sensing ability through CEST method, and which can also be combined with BIRDS detection scheme.

In various embodiments, the methods described herein include measuring pH and/or temperature sensitivity study in vitro or in vivo.

In various embodiments, the measuring of the CEST of —NH protons in the compound includes with the mapping of extracellular pH of a cancer tumor.

In various embodiments, compounds of the disclosure such as TnNOTP⁴⁻, TnDOTP⁶⁻, TnDOTA-4AMP⁶⁻ are agents for sodium ion sensing ability (Table 2).

In various embodiments, compounds of the disclosure such as wherein the measuring of the volume of compartmentalized sodium ions includes a volume of extracellular sodium concentration due to interaction with compounds of formula (Ia), (Ib), (Ic), (Id), or (Ie) for a cancer diagnosis and prognosis.

In various embodiments, the methods described herein are suitable for identification of tumor type, demarcation of tumor boundary, prognosis of tumor therapy.

In various embodiments, compounds of the disclosure can be used for the simultaneous mapping of sodium and pH of a cancerous tumor.

### Administration/Dosage/Formulations

In various embodiments, the compounds of the disclosure are administered in a diagnostically effective amount. The dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to accurately image a tissue volume in in the patient. A diagnostically effective amount of the therapeutic compound necessary to achieve a therapeutic effect may vary according to factors such as the state of the disease or disorder in the patient; the age, sex, and weight of the patient; and the ability of the therapeutic compound to accurately image a tissue volume in in the patient.

Actual dosage levels of the active ingredients in the pharmaceutical compositions described herein may be varied so as to obtain an amount of the active ingredient that is effective to achieve the desired imaging for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of compound calculated to produce the desired effect in association with the required pharmaceutical vehicle.

In certain embodiments, the compositions described herein are formulated using one or more pharmaceutically acceptable excipients or carriers. In certain embodiments, the pharmaceutical compositions described herein comprise a diagnostically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a diagnostically effective amount of a compound described herein.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In various embodiments, compounds of formula (Ia), (Ib), (Ic), (Id), or (Ie) are administered in a single intravenous injection, which can be a rapid injection (the entire dose administered in less than 15, 30, or 60 seconds) or an infusion over time (over 1 to 30 minutes).

It should be understood that the formulations and compositions described herein are not limited to the particular formulations and compositions that are described herein.

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution, and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as such as lauryl, stearyl, or oleyl alcohols, or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Examples

Various embodiments of the present application can be better understood by reference to the following Examples which are offered by way of illustration. The scope of the present application is not limited to the Examples given herein.

### Synthesis of Compounds of the Disclosure

The method of complex synthesis is carried out in an organic solvent so that divalent transition metal ions (Tn²⁺=Fe²⁺, Co²⁺ or Ni²⁺) cannot be oxidized. About 0.2 mM a ligand (NOTP⁶⁻, DOTP⁸⁻, or DOTA-4AMP⁸⁻) is dissolved in absolute ethanol in an Erlenmeyer flask and purged with inert nitrogen gas. About 0.2 mM of a transition metal salt [Ni(NO₃)₂6H₂O, Co(NO₃)₂6H₂O or FeSO₄·7H₂O] is dissolved in absolute ethanol under inert atmosphere of nitrogen gas in a separate Erlenmeyer flask. Finally respective ligand solution is mixed with respective salt solution and stirred under inert atmosphere for 24 h. After completion of the synthesis, the complex is precipitated with excess diethyl ether and washed three times to remove uncomplexed Tn²⁺. The precipitate is dried at room temperature. Finally, dried powders are dissolved in water and the pH of solution is maintained to 7 using HCl and KOH. The pH-maintained samples are lyophilized and stored at −20 °C for further characterization and evaluation. The samples are further purified and analyzed on a HPCL using C18 column. The transition metal ion concentration of the complexes is then confirmed by ICP-MS analysis.

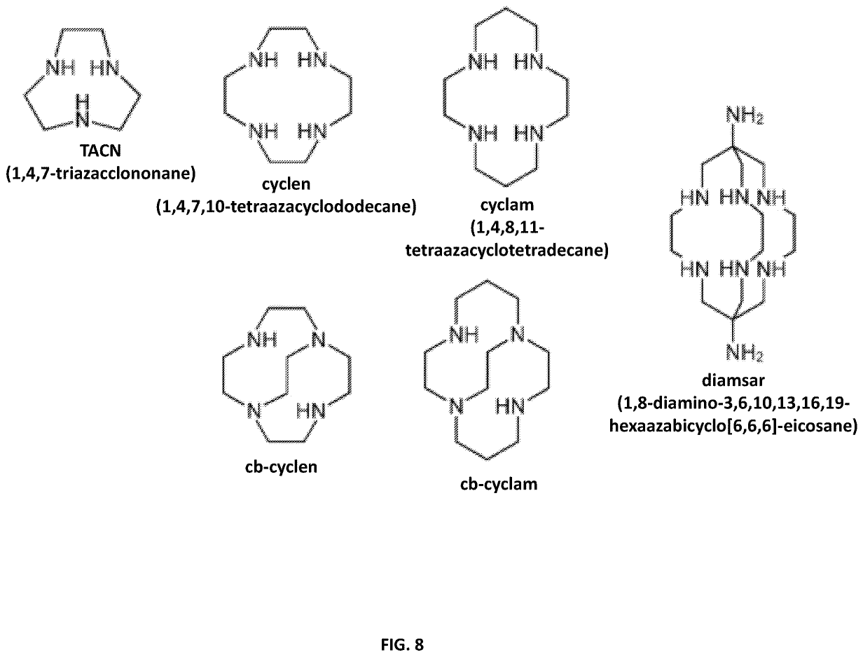

While complexation with Tn²⁺ and ligands like TACN (1,4,7-triazacyclononane) and cyclen (1,4,7,10-tetraazacyclododecane) are demonstrated here, similar complexation can be achieved with Tn²⁺ and other ligands, e.g., cyclam (1,4,8,11-tetraazacyclotetradecane) and their cross bridged (cb) versions like cb-cyclen and cb-cyclam or even diamsar (1,8-diamino-3,6,10,13,16,19-hexaazabicyclo[6,6,6]-eicosane) as shown in FIG. 8.

### NMR Study:

The synthesized complexes are characterized for water proton relaxivities with different concentrations of complexes, as well as their proton sensing (with BIRDS and CEST) and sodium sensing (with ²³Na-MRSI) properties on a Bruker AVANCE III HD 500 vertical-bore spectrometer (Bruker, Billerica, MA, USA) interfaced with Bruker TopSpin v3.2 software.

### BIRDS Characterization:

The ¹H NMR chemical shifts for TnNOTP⁴⁻, TnDOTP⁶⁻, and TnDOTA-4AMP⁶⁻ complexes (10 mM, pH ranges 6.2-8.0) are obtained by acquiring proton spectra at various temperatures (298 K to 318 K) and calibrated on a Bruker spectrometer (Billerica, MA) at 11.7 T. All spectra are line broadened (100 Hz), phased, and baseline corrected. The chemical shifts of proton resonances are measured relative to the water resonance (4.7 ppm). Temperature and pH sensitive non-exchangeable proton resonances in each complex are chosen for BIRDS detection. The temperature and pH dependences of proton chemical shifts are fitted to second-order polynomial equations:

δ=a+b pH+cT+dpH²+eT²+f pHT Eq. 1

Where, the coefficients a-f are obtained from the fit. The relaxation times (T1 and T2) of these resonances are measured at 35 °C using typical inversion-recovery and spin-echo methods, respectively.

### CEST Characterization:

All CEST experiments for TnDOTA-4AMP⁶⁻ complexes (20 mM, pH range 6.2-8.0) are collected with a continuous wave saturation RF pulse (4 s, 30 μT) over a range of frequencies (±150 ppm, 0.5 ppm each step) followed by a short observe RF pulse to measure the residual water signal at different temperatures (298 K to 318 K) on a Bruker spectrometer at 11.7 T. The CEST properties are analyzed by calculating the Z-spectra (i.e. plots of normalized bulk water signal intensity [Mₛ/M₀] as a function of saturation frequency). The CEST effect is quantified as a decrease in total bulk water intensity according to the following equation:

$$CEST = 1 - \frac{{M}_{s}}{{M}_{0}} \qquad Eq . 2$$

Where, Mₛ is the magnetization for saturation at the frequency of interest and M₀ is the reference magnetization for saturation at a frequency further away from resonance of interest. The second term on the right-hand side of Eq. 2 is inversely proportional to (1+kT₁), where k is the exchange rate and T₁ is the bulk water longitudinal relaxation time, and k is determined by fitting the Z-spectra to Bloch equations modified for chemical exchange.

### ²³Na Spectra Acquisition:

Sodium-23 (²³Na), which has 100% natural abundance, is also visible by magnetic resonance (I=3/2) and provides the second-strongest signal among biological ions after proton. Gyromagnetic ratio of ²³Na (γNa=11.26 MHz/T) is ~¼ that of ¹H, but with shorter T₁ and T₂, so under idealized situations the sensitivity for ²³Na-MRI may potentially be comparable. Sodium (Na⁺) concentration is normally low intracellularly (~10 mM) and high in blood and extracellular spaces (~150 mM), producing a strong transmembrane Nat gradient (ΔNa⁺ₘₑₘ≈140 mM) and a weak transendothelial Na⁺ gradient (ΔNa⁺_{end}≈0 mM). Distributions of sodium ion (Na⁺) across biological membranes are tightly regulated, and thus variations of Nat across compartments often imply pathological states.

In vitro experiments are performed using concentric NMR tubes (spherical and cylindrical; Wilmad-LabGlass, Vineland, NJ, USA). One compartment (inner) contained 150 mM NaCl and the other (outer) contained the same but with varying amounts of transition metal complexes and 10% v/v ²H₂O to lock the spectrometer frequency using the ²H₂O signal. Each solution is pH-adjusted using HCl or KOH to give five different pH values. ²³Na-NMR spectra are collected on the same Bruker system as for ¹H NMR. A single ²³Na square pulse is used to globally excite the volume of interest (repetition time TR=275 ms) collecting 2048 FID points in the time domain with an acquisition time t_{aq}=38.9 ms, averaged 1024 times. Spectra are analyzed applying 10 Hz line broadening and manual zeroth- and first-order phasing. The chemical shift and line width of the shifted peak are measured using the “peakw” function on TopSpin.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following enumerated embodiments are provided, the numbering of which is not to be construed as designating levels of importance:

Embodiment 1: A compound, a deuterated analogue thereof, or a pharmaceutically acceptable salt thereof, of formula:

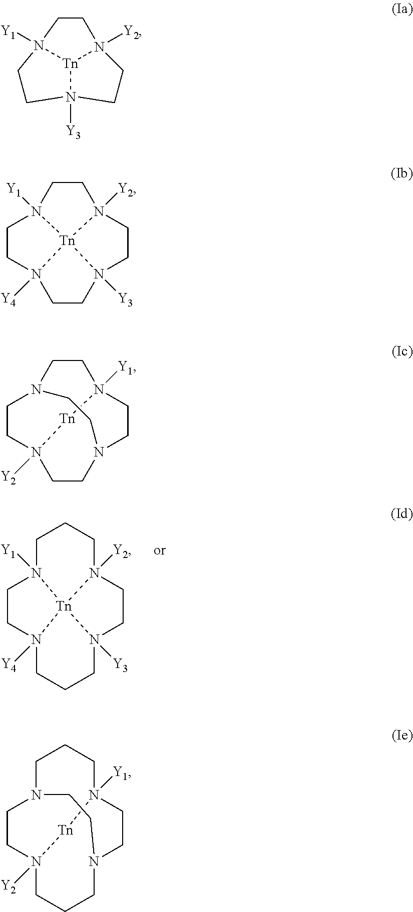

wherein:
each Tn is independently a divalent or trivalent transition metal ion selected from the group consisting of Mn, Fe, Co, Ni, and Zn;
each Y₁, Y₂, Y₃, or Y₄ is independently H or —X—CH₂—P(O)(OH)₂;
each X is independently a bond or LL;
each LL is independently a bivalent, saturated or unsaturated, straight or branched C₁-12 hydrocarbon chain, wherein 0-6 methylene units of the hydrocarbon are independently replaced with —O—, —S—, —N(R*)—, —OC(═O)—, —C(═O)O—, —S(O)—, —S(O)₂—, —N(R*)S(O)₂—, —S(O)₂N(R*)—, —N(R*)C(═O)—, —C(═O)N(R*)—, —OC(═O)N(R*)—, or —N(R*)C(═O)O—,
   wherein each R* is independently hydrogen, C₁₋₆ alkyl, or C₃₋₆ cycloalkyl;
with the proviso that at least two of Y₁, Y₂, Y₃, and Y₄, if present, are independently —X—CH₂—P(O)(OH)₂.

Embodiment 2: The compound of Embodiment 2, wherein in (Ia), (Ib), or (Id) at least three of Y₁, Y₂, Y₃, and Y₄, if present, are independently —X—CH₂—P(O)(OH)₂.

Embodiment 3: The compound of any one of Embodiments 1-2, wherein X is

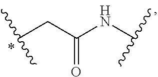 [SMILES: *CC(=O)N*]

and
wherein the * designates the point of attachment to the ring N atom in formula (Ia), (Ib), (Ic), (Id), or (Ie).

Embodiment 4: The compound of any one of Embodiments 1-3, wherein the Tn is in the +2 oxidation state.

Embodiment 5: The compound of any one of Embodiments 1-4, wherein Tn is Fe. Co, or Ni.

Embodiment 6: The compound of any one of Embodiments 1-5, which has the structure

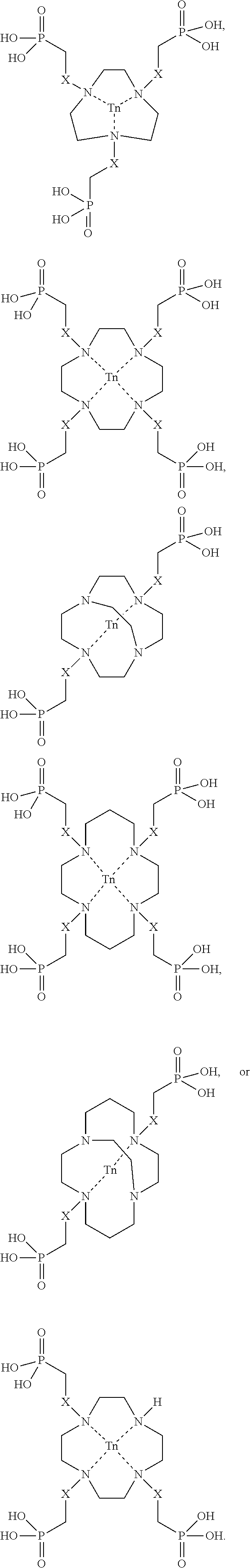

Embodiment 7: The compound of any one of Embodiments 1-6, which has the structure

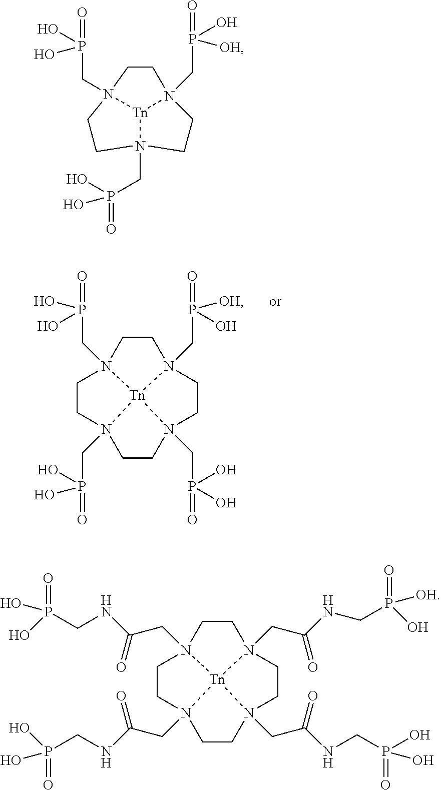

Embodiment 8: A pharmaceutical composition comprising the compound of any one of Embodiments 1-7 and at least one pharmaceutically acceptable carrier or excipient.

Embodiment 9: A method of imaging a tissue volume using a magnetic resonance imaging (MRI), the method comprising:
contacting a bolus of a contrast agent comprising the compound of any one of Embodiments 1-7 with the tissue volume; and
acquiring magnetic resonance image data, wherein the image data comprises at least one of chemical shift data for non-exchangeable protons in the contrast agent in and surrounding the tissue volume, extracellular sodium concentration surrounding the tissue volume, and extracellular pH data surrounding the tissue volume.

Embodiment 10: The method of Embodiment 9, wherein the tissue volume comprises a cancerous tumor.

Embodiment 11: The method of Embodiment 10, wherein the acquiring magnetic resonance image data comprises measuring of the chemical exchange saturation transfer (CEST) of NH protons in the contrast agent and mapping of extracellular pH of the cancerous tumor.

Embodiment 12: The method of any one of Embodiments 9-11, wherein the acquiring magnetic resonance data comprises:
a) exposing the contrast agent to a saturation radiofrequency (RF) pulse;
b) exposing the contrast agent to at least one sampling RF pulse; and
c) exposing the contrast agent to one or more RF pulses to obtain CEST data.

Embodiment 13: The method of any one of Embodiments 9-12, wherein the tissue volume comprises an in vitro tissue sample.

Embodiment 14: The method of any one of Embodiments 9-13, wherein the tissue volume comprises in vivo tissue in a mammal.

Embodiment 15: The method of claim 14, wherein the mammal is a human.

Embodiment 16: A method for monitoring the efficacy of a cancer treatment in a patient undergoing chemotherapy, the method comprising:
administering a bolus of a contrast agent comprising the compound of any one of Embodiments 1-7 to the patient; and acquiring magnetic resonance image data, wherein the image data comprises at least one of chemical shift data for non-exchangeable protons in the contrast agent in and surrounding the tissue volume, extracellular sodium concentration surrounding the tissue volume, and extracellular pH data surrounding the tissue volume.

Embodiment 17: The method of Embodiment 16, wherein the tissue volume comprises a cancerous tumor.

Embodiment 18: The method of Embodiment 17, wherein the acquiring magnetic resonance image data comprises measuring of the CEST of NH protons in the contrast agent and mapping of extracellular pH of the cancerous tumor.

Embodiment 19: The method of any one of Embodiments 16-18, wherein the acquiring magnetic resonance data comprises:
a) exposing the contrast agent to a saturation radiofrequency (RF) pulse;
b) exposing the contrast agent to at least one sampling RF pulse; and
c) exposing the contrast agent to one or more RF pulses to obtain CEST data.

Embodiment 20: A method for monitoring or determining a cancer prognosis in a patient, the method comprising:
administering a bolus of a contrast agent comprising the compound of any one of Embodiments 1-7 to the patient, wherein the patient has at least one tissue volume comprising cancerous tissue; and
acquiring magnetic resonance image data,
wherein the image data comprises extracellular pH data surrounding the tissue volume.

Embodiment 21: The method of Embodiment 20, wherein a greater volume of measured acidic extracellular pH in an environment surrounding the tissue volume is correlated with a more aggressive cancer.

Embodiment 22: The method of any one of Embodiments 20-21, wherein the acquiring magnetic resonance image data comprises measuring of the CEST of NH protons in the contrast agent and mapping of extracellular pH of the cancerous tissue.

Embodiment 23: The method of any one of Embodiments 20-22, wherein the acquiring magnetic resonance data comprises:
a) exposing the contrast agent to a saturation radiofrequency (RF) pulse;
b) exposing the contrast agent to at least one sampling RF pulse; and
c) exposing the contrast agent to one or more RF pulses to obtain CEST data.

Embodiment 24: A method of making a compound, a deuterated analog thereof, or a pharmaceutically acceptable salt thereof, or formula:

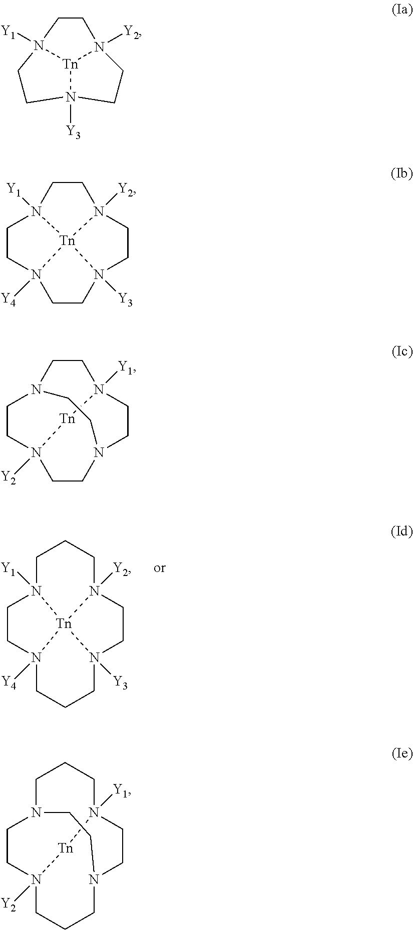

wherein:
each Tn is independently a divalent or trivalent transition metal ion selected from the group consisting of Mn, Fe, Co, Ni, and Zn;
each Y₁, Y₂, Y₃, or Y₄ is independently H or —X—CH₂—P(O)(OH)₂;
each X is independently a bond or LL;
each LL is independently a bivalent, saturated or unsaturated, straight or branched C₁₋₁₂ hydrocarbon chain, wherein 0-6 methylene units of the hydrocarbon are independently replaced with —O—, —S—, —N(R*)—, —OC(═O)—, —C(═O)O—, —S(O)—, —S(O)₂—, —N(R*)S(O)₂—, —S(O)₂N(R*)—, —N(R*)C(═O)—, —C(═O)N(R*)—, —OC(═O)N(R*)—, or —N(R*)C(═O)O—,
   wherein each R* is independently hydrogen, C₁₋₆ alkyl, or C₃₋₆ cycloalkyl;
      with the proviso that at least two of Y₁, Y₂, Y₃, and Y₄, if present, are independently —X—CH₂—P(O)(OH)₂;
the method comprising contacting a salt of Tn with a ligand, or a salt thereof, having the structure

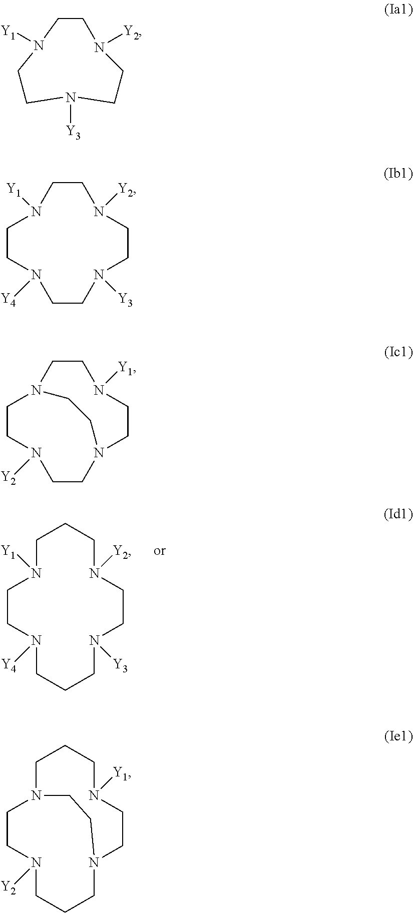

respectively, in an organic solvent under inert atmosphere to produce the compound.

Embodiment 25: The method of Embodiment 24, wherein the solvent is ethanol.

Embodiment 26: The method of any one of Embodiments 24-25, wherein the ligand is an alkali metal salt or alkaline earth salt.

Embodiment 27: The method of any one of Embodiments 24-26, wherein the salt of Tn is a fluoride, chloride, bromide, or nitrate salt.

1. A compound, a deuterated analogue thereof, or a pharmaceutically acceptable salt thereof, of formula: 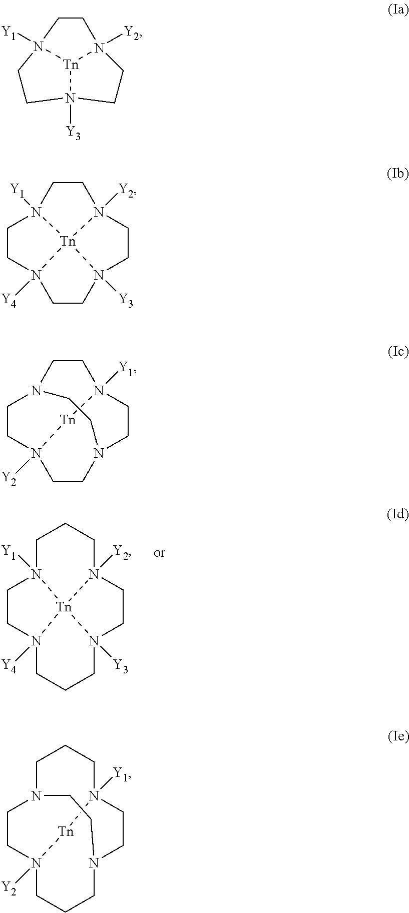
wherein:
each Tn is independently a divalent or trivalent transition metal ion selected from the group consisting of Mn, Fe, Co, Ni, and Zn;
each Y₁, Y₂, Y₃, or Y₄ is independently H or —X—CH₂—P(O)(OH)₂;
each X is independently a bond or LL;
each LL is independently a bivalent, saturated or unsaturated, straight or branched C₁₋₁₂ hydrocarbon chain, wherein 0-6 methylene units of the hydrocarbon are independently replaced with —O—, —S—, —N(R*)—, —OC(═O)—, —C(═O)O—, —S(O)—, —S(O)₂—, —N(R*)S(O)₂—, —S(O)₂N(R*)—, —N(R*)C(═O)—, —C(═O)N(R*)—, —OC(═O)N(R*)—, or —N(R*)C(═O)O—,
wherein each R* is independently hydrogen, C₁₋₆ alkyl, or C₃₋₆ cycloalkyl;
with the proviso that at least two of Y₁, Y₂, Y₃, and Y₄, if present, are independently —X—CH₂—P(O)(OH)₂.

2. The compound of claim 1, wherein in (Ia), (Ib), or (Id) at least three of Y₁, Y₂, Y₃, and Y₄, if present, are independently —X—CH₂—P(O)(OH)₂.

3. The compound of claim 1, wherein X is 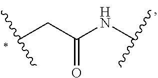 [SMILES: *CC(=O)N*]
and
wherein the * designates the point of attachment to a ring N atom in formula (Ia), (Ib), (Ic), (Id), or (Ie).

4. The compound of claim 1, wherein at least one of the following applies:
(a) the Tn is in a (+2) oxidation state;
(b) Tn is Fe, Co, or Ni.

5. (canceled)

6. The compound of claim 1, which has the structure:

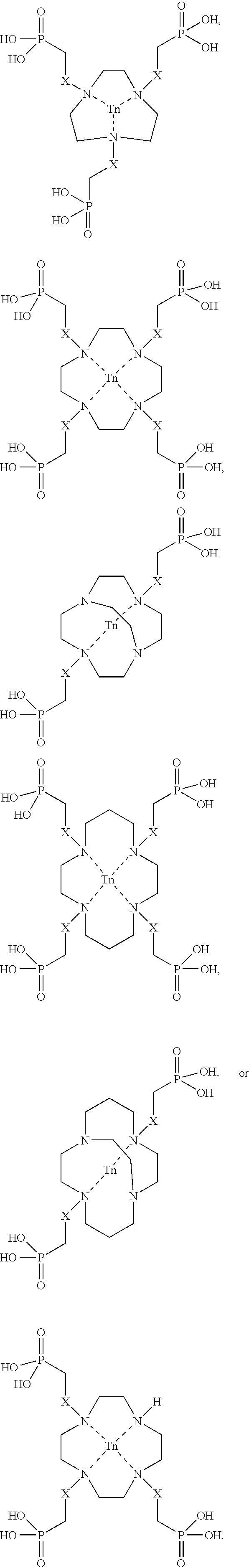

7. The compound of claim 1, which has the structure:

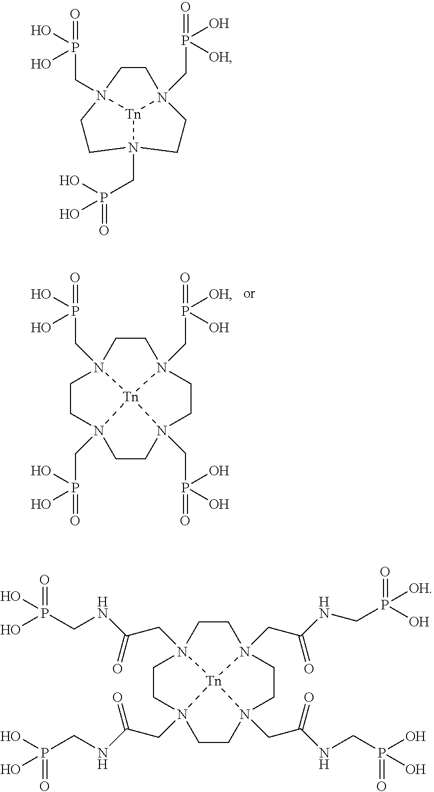

8. A pharmaceutical composition comprising the compound of claim 1 and at least one pharmaceutically acceptable carrier or excipient.

9. A method of imaging a tissue volume using a magnetic resonance imaging (MRI), the method comprising:
contacting a bolus of a contrast agent comprising the compound of claim 1 with the tissue volume; and
acquiring magnetic resonance image data, wherein the image data comprises at least one of chemical shift data for non-exchangeable protons in the contrast agent in and surrounding the tissue volume, extracellular sodium concentration surrounding the tissue volume, and extracellular pH data surrounding the tissue volume.

10. The method of claim 9, wherein at least one of the following applies:
(a) the tissue volume comprises a cancerous tumor;
(b) the tissue volume comprises an in vitro tissue sample;
(c) the tissue volume comprises in vivo tissue in a mammal, optionally wherein the mammal is a human.

11. The method of claim 9, wherein at least one of the following applies:
(a) the acquiring magnetic resonance image data comprises measuring of the chemical exchange saturation transfer (CEST) of NH protons in the contrast agent and mapping of extracellular pH of the cancerous tumor;
(b) the acquiring magnetic resonance data comprises:
i) exposing the contrast agent to a saturation radiofrequency (RF) pulse;
ii) exposing the contrast agent to at least one sampling RF pulse; and
iii) exposing the contrast agent to one or more RF pulses to obtain CEST data.

12-15. (canceled)

16. A method for monitoring the efficacy of a cancer treatment in a patient undergoing chemotherapy, the method comprising:
administering a bolus of a contrast agent comprising the compound of claim 1 to the patient; and
acquiring magnetic resonance image data, wherein the image data comprises at least one of chemical shift data for non-exchangeable protons in the contrast agent in and surrounding a tissue volume, extracellular sodium concentration surrounding the tissue volume, and extracellular pH data surrounding the tissue volume.

17. The method of claim 16, wherein the tissue volume comprises a cancerous tumor.

18. The method of claim 16, wherein at least one of the following applies:
(a) the acquiring magnetic resonance image data comprises measuring of the CEST of NH protons in the contrast agent and mapping of extracellular pH of the cancerous tumor;
(b) the acquiring magnetic resonance data comprises:
i) exposing the contrast agent to a saturation radiofrequency (RF) pulse;
ii) exposing the contrast agent to at least one sampling RF pulse; and
iii) exposing the contrast agent to one or more RF pulses to obtain CEST data.

19. (canceled)

20. A method for monitoring or determining a cancer prognosis in a patient, the method comprising:
administering a bolus of a contrast agent comprising the compound of claim 1 to the patient, wherein the patient has at least one tissue volume comprising cancerous tissue; and
acquiring magnetic resonance image data,
wherein the image data comprises extracellular pH data surrounding the tissue volume.

21. The method of claim 20, wherein a greater volume of measured acidic extracellular pH in an environment surrounding the tissue volume is correlated with a more aggressive cancer.

22. The method of claim 20, wherein at least one of the following follows:
(a) the acquiring magnetic resonance image data comprises measuring of the CEST of NH protons in the contrast agent and mapping of extracellular pH of the cancerous tissue;
(b) the acquiring magnetic resonance data comprises:
i) exposing the contrast agent to a saturation radiofrequency (RF) pulse;
ii) exposing the contrast agent to at least one sampling RF pulse; and
iii) exposing the contrast agent to one or more RF pulses to obtain CEST data.

23. (canceled)

24. A method of making a compound, a deuterated analog thereof, or a pharmaceutically acceptable salt thereof, or formula: 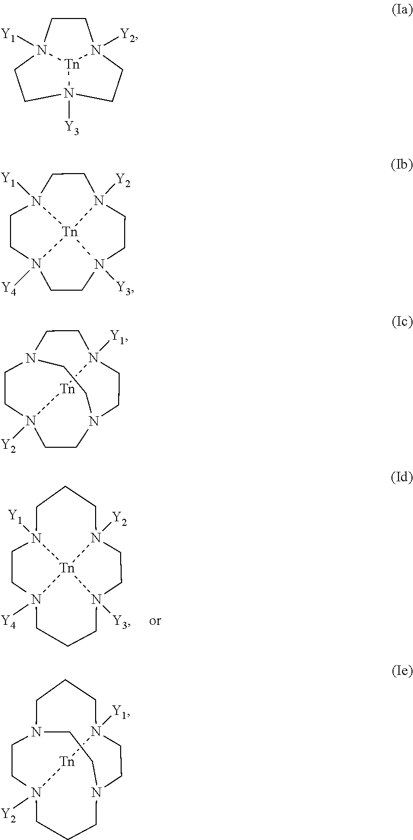
wherein:
each Tn is independently a divalent or trivalent transition metal ion selected from the group consisting of Mn, Fe, Co, Ni, and Zn;
each Y₁, Y₂, Y₃, or Y₄ is independently H or —X—CH₂—P(O)(OH)₂;
each X is independently a bond or LL;
each LL is independently a bivalent, saturated or unsaturated, straight or branched C₁₋₁₂ hydrocarbon chain, wherein 0-6 methylene units of the hydrocarbon are independently replaced with —O—, —S—, —N(R*)—, —OC(═O)—, —C(═O)O—, —S(O)—, —S(O)₂—, —N(R*)S(O)₂—, —S(O)₂N(R*)—, —N(R*)C(═O)—, —C(═O)N(R*)—, —OC(═O)N(R*)—, or —N(R*)C(═O)O—,
wherein each R* is independently hydrogen, C₁₋₆ alkyl, or C₃₋₆ cycloalkyl;
with the proviso that at least two of Y₁, Y₂, Y₃, and Y₄, if present, are independently —X—CH₂—P(O)(OH)₂;
the method comprising contacting a salt of Tn with a ligand, or a salt thereof, having the structure 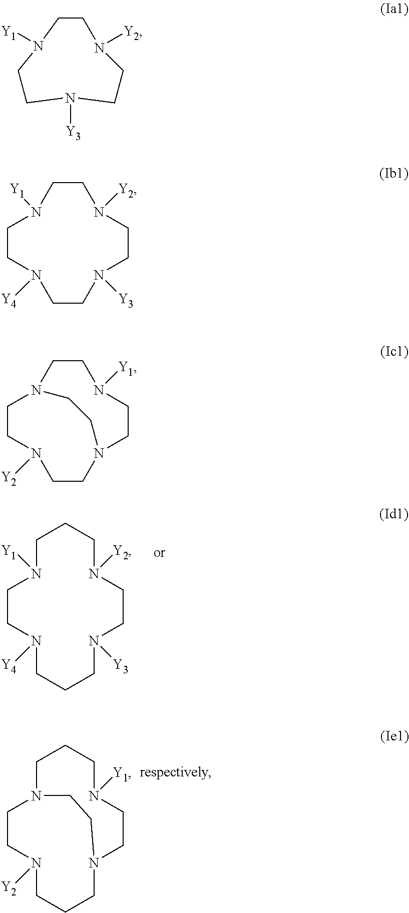
in an organic solvent under inert atmosphere to produce the compound.

25. The method of claim 24, wherein at least one of the following applies:
(a) the solvent is ethanol;
(b) the ligand is an alkali metal salt or alkaline earth salt;
(c) the salt of Tn is a fluoride, chloride, bromide, or nitrate salt.

26-27. (canceled)
